SEQUENCE LISTING (80 sequences)

SEQ ID NO: 1 - protein, 36 aa, Chlamydia virus Chp1
MVRRRRLRRRISRRIFRRTVARVGRRRRSFRGGIRF

SEQ ID NO: 2 - protein, 44 aa, Chlamydia phage 2
MRLKMARRRYRLPRRRSRRLFSRTALRMHPRNRLRRIMRGGIRF

SEQ ID NO: 3 - protein, 35 aa, Chlamydia virus CPAR39
MCKKVCKKCPKKGPKNAPKIGAFYERKTPRLKQST

SEQ ID NO: 4 - protein, 39 aa, Chlamydia phage 4
MARRYRLSRRRSRRLFSRTALRMHRRNRLRRIMRGGIRF

SEQ ID NO: 5 - protein, 39 aa, Artificial Sequence
  Description of Artificial Sequence Synthetic polypeptide
MAEQYELSQEQSEQLFSETALQMHEQNELQEIMQGGIEF

SEQ ID NO: 6 - protein, 40 aa, Guinea pig Chlamydia phage
MARRRYRLPRRRSRRLFSRTALRMHPRNRLRRIMRGGIRF

SEQ ID NO: 7 - protein, 38 aa, Chlamydia trachomatis
MKRRKMTRKGSKRLFTATADKTKSINTAPPPMRGGIRL

SEQ ID NO: 8 - protein, 39 aa, Chlamydia trachomatis
MSKKRSRMSRRRSKKLFSKTALRTKSVNTRPPMRGGFRF

SEQ ID NO: 9 - protein, 40 aa, Chlamydia trachomatis
MSLRRHKLSRKASKRIFRKGASRTKTLNTRATPMRGGFRI

SEQ ID NO: 10 - protein, 38 aa, Chlamydia trachomatis
MKRRKLSKKKSRKIFTRGAVNVKKRNLRARPMRGGFRI

SEQ ID NO: 11 - protein, 37 aa, Chlamydia trachomatis
MAKKMTKGKDRQVFRKTADRTKKLNVRPLLYRGGIRL

SEQ ID NO: 12 - protein, 39 aa, Chlamydia trachomatis
MAGKKMVSKGKDRQIFRKTADRTKKMNVRPLLYRGGIRL

SEQ ID NO: 13 - protein, 41 aa, Marine gokushovirus
MRRPRKMNYKKSKRMFSRTAARTHRKNSLRGSRPMRGGIRL

SEQ ID NO: 14 - protein, 34 aa, Gokushovirinae Fen672_31
MSKKASRKSFTKGAVKVHKKNVPTRVPMRGGIRL

SEQ ID NO: 15 - protein, 35 aa, Unknown
  Description of Unknown Microviridae phage sequence
MKMRKRTDKRVFTRTAAKSKKVNIAPKIFRGGIRL

SEQ ID NO: 16 - protein, 39 aa, Escherichia coli
MARSRRRMSKRSSRRSFRKYAKTHKRNFKARSMRGGIRL

SEQ ID NO: 17 - protein, 47 aa, Cognatishimia maritima
MESPNSRSQLGITLYLLSTIFPDACFRYRRELPYPLVIWGVATLCLQ

SEQ ID NO: 18 - protein, 39 aa, Escherichia coli
MARSRRRMSKRSSRRSFRKYAKSHKKNFKARSMRGGIRL

SEQ ID NO: 19 - protein, 37 aa, Oscillibacter sp.
MRKRMSKRVDKKVFRRTAASAKKINIDPKIYRGGIRL

SEQ ID NO: 20 - protein, 37 aa, Unknown
  Description of Unknown Microviridae phage sequence
MRRRRLSRRTSRRFFRKGLKVRRRNLRARPMRGGFRI

SEQ ID NO: 21 - protein, 39 aa, Unknown
  Description of Unknown Microviridae phage sequence
MARRKKMKGKRDKRVFKQTANKTKAINISPKNMRGGTRL

SEQ ID NO: 22 - protein, 53 aa, Marine gokushovirus
MLTVWSDTPTIKRRKDMYRKRMSRKKSKKVFAKTAMKVNKRNHVKPMRGGYRI

SEQ ID NO: 23 - protein, 39 aa, Marine gokushovirus
MMKYRKKMSAKSSRKQFTKGAMKVKGKNFTKPMRGGIRL

SEQ ID NO: 24 - protein, 38 aa, Marine gokushovirus
MRRYNVNKGKSAKKFRKQVSKTKVANLRSNPMRGGWRL

SEQ ID NO: 25 - protein, 28 aa, Spiroplasma virus
MAYRGFKTSRVVKHRVRRRWFNHRRRYR

SEQ ID NO: 26 - protein, 38 aa, Spiroplasma virus
MRRKVKNTKRHQWRLTHSARSIKRANIMPSNPRGGRRF

SEQ ID NO: 27 - DNA, 111 bp, Chlamydia virus Chp1
atggttcgtagaagacgtttgagaagaagaataagtagaagaatttttagaagaacagtagctagagttggtagaaggcgaaggtcttttcgtggtggtattagattttaa

SEQ ID NO: 28 - DNA, 135 bp, Chlamydia phage 2
atgaggttaaaaatggcacgaagaagatacagacttccgcgacgtagaagtcgaagacttttttcaagaactgcattgaggatgcatccaagaaataggcttcgaagaattatgcgtggcggcattaggttctag

SEQ ID NO: 29 - DNA, 108 bp, Chlamydia virus CPAR39
ttgtgcaaaaaagtgtgcaaaaaatgcccaaaaaaagggccaaaaaatgcccccaaaatcggagcattttacgagagaaaaacacctagacttaaacagtctacttga

SEQ ID NO: 30 - DNA, 120 bp, Chlamydia phage 4
atggcacgaagatacagactttcgcgacgcagaagtcgacgacttttttcaagaactgcattaagaatgcatcgaagaaatagacttcgaagaattatgcgtggcggcattaggttttag

SEQ ID NO: 31 - DNA, 120 bp, Artificial Sequence
  Description of Artificial Sequence Synthetic polynucleotide
atggcggaacagtatgaactgagccaggaacagagcgaacagctgtttagcgaaaccgcgctgcagatgcatgaacagaacgaactgcaggaaattatgcagggcggcattgaattttaa

SEQ ID NO: 32 - DNA, 123 bp, Guinea pig Chlamydia phage
atggcacgaagaagatacagacttccgcgacgtagaagtcgaagacttttttcaagaactgcattaaggatgcatccaagaaataggcttcgaagaattatgcgtggcggcattaggttctag

SEQ ID NO: 33 - DNA, 117 bp, Chlamydia trachomatis
atgaaacgtagaaaaatgacaagaaaaggttctaagcgtctttttactgcaactgctgataaaactaaatctatcaatactgccccgccgccaatgcgtggcggtatccggttgtaa

SEQ ID NO: 34 - DNA, 120 bp, Chlamydia trachomatis
atgtctaaaaagcgttctcgcatgtctcgccgccgttctaagaagttgttctcgaaaacggctctccgcacgaagagtgtcaacacccgtccgcctatgcgcggagggttccggttctga

SEQ ID NO: 35 - DNA, 123 bp, Chlamydia trachomatis
atgtctcttcgtcgtcataagctttctcgtaaggcgtctaagcgtatttttcgtaaaggtgcatcacgcacgaagactttgaatactcgtgctacgcctatgcgcggcggtttccgtatttaa

SEQ ID NO: 36 - DNA, 117 bp, Chlamydia trachomatis
atgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 37 - DNA, 114 bp, Chlamydia trachomatis
atggctaaaaaaatgactaaaggcaaggatcgtcaggtttttcgtaaaaccgctgatcgtactaagaaactcaatgttagaccgttgttatatcgaggaggtatcagattatga

SEQ ID NO: 38 - DNA, 120 bp, Chlamydia trachomatis
atggcaggaaaaaaaatggtatcaaaaggaaaagatagacagattttccgaaaaactgctgatcgcactaaaaaaatgaatgtgcgcccgctattatatcgtggaggtattagattatga

SEQ ID NO: 39 - DNA, 126 bp, Marine gokushovirus
atgagaagaccaagaaaaatgaactataaaaaatcaaaaagaatgttttcacgcacagcagcgagaacacacagaaaaaactctctaagaggtagccgacctatgagaggcggaatacgtctttaa

SEQ ID NO: 40 - DNA, 105 bp, Gokushovirinae Fen672_31
atgtcgaagaaggcgtcgaggaagagttttactaagggtgccgttaaggttcataagaaaaatgttcctactcgtgttcctatgcgtggcggtattaggctttag

SEQ ID NO: 41 - DNA, 108 bp, Unknown
  Description of Unknown Microviridae phage sequence
atgaaaatgcgtaagcggacggacaagcgagtgtttacccgcaccgctgctaagtccaagaaagtgaacattgccccgaaaatttttagaggaggtatccgtctgtga

SEQ ID NO: 42 - DNA, 120 bp, Escherichia coli
atggctcgttctcgccgtcgtatgtccaagcgttcttcccgtcgttcgttccgtaagtacgcaaagacgcataaacgtaactttaaagcccgctctatgcgtggtggaattcgtctttga

SEQ ID NO: 43 - DNA, 144 bp, Cognatishimia maritima
atggaaagcccgaacagccgcagccagctgggcattaccctgtatctgctgagcaccatttttccggatgcgtgctttcgctatcgccgcgaactgccgtatccgctggtgatttggggcgtggcgaccctgtgcctgcagtaa

SEQ ID NO: 44 - DNA, 120 bp, Escherichia coli
atggctcgttcccgtagacgtatgtctaagcgttcttcccgccgttcgttccgcaagtatgcgaagtcgcataagaagaactttaaagcccgctcaatgcgtggcggtatccgtttataa

SEQ ID NO: 45 - DNA, 114 bp, Oscillibacter sp.
atgagaaagcgaatgtctaagcgtgttgacaagaaggtgttccgtcgtactgccgcatctgccaagaagattaacattgaccccaagatttaccgtggaggtattcgcctatga

SEQ ID NO: 46 - DNA, 114 bp, Unknown
  Description of Unknown Microviridae phage sequence
atgagacgtcgtcgtctatcccgcagaacttcccgccgttttttccgtaaaggacttaaggttcgccgtcgtaacctccgcgcgagacccatgagaggcggattcagaatttga

SEQ ID NO: 47 - DNA, 120 bp, Unknown
  Description of Unknown Microviridae phage sequence
atggcacgacgcaagaagatgaaaggcaagcgggataaacgggtgtttaagcagacagccaacaaaaccaaggctatcaacatcagcccaaaaaacatgagagggggtacgagactgtga

SEQ ID NO: 48 - DNA, 162 bp, Marine gokushovirus
atgttaactgtgtggagtgacacccctaccataaaaaggagaaaagacatgtatagaaagagaatgtcaagaaagaaaagtaaaaaggtttttgcaaaaaccgcaatgaaagtaaataaaagaaaccacgttaaacctatgcgtggtggatatagaatataa

SEQ ID NO: 49 - DNA, 120 bp, Marine gokushovirus
atgatgaagtacagaaaaaaaatgagcgctaaaagtagccgaaagcaatttacaaaaggcgccatgaaagtgaagggtaaaaacttcacaaaaccaatgcgcggaggcatccgtctatag

SEQ ID NO: 50 - DNA, 117 bp, Marine gokushovirus
atgcgacgttacaatgtaaataaaggtaaatctgctaagaagtttcgaaagcaggtaagtaagacgaaggttgcaaacctacgttctaatccaatgcgaggtggttggagactctaa

SEQ ID NO: 51 - DNA, 87 bp, Spiroplasma virus
atggcttatcgtggttttaaaacgagtcgtgttgtaaaacatagagtacgtagaagatggtttaatcatagaagacgttatagatag

SEQ ID NO: 52 - DNA, 117 bp, Spiroplasma virus
gtgagacgcaaggttaagaacacaaagcgtcatcagtggaggttgactcattctgcacgttcaattaaacgtgctaatataatgccgtcaaatcctcgtggtggacgtcgtttttag

SEQ ID NO: 53 - DNA, 135 bp, Chlamydia phage 3
atgaggttaaaaatggcacgaagaagatacagacttccgcgacgtagaagtcgaagacttttttcaagaactgcattaaggatgcatccaagaaataggcttcgaagaattatgcgtggcggcattaggttctag

SEQ ID NO: 54 - protein, 44 aa, Chlamydia phage 3; identical to SEQ ID NO: 2

SEQ ID NO: 55 - protein, 39 aa, Escherichia sp.
MARSRRRMSKRSSRRSFRKYAKTHKKNFKARSMRGGIRL

SEQ ID NO: 56 - protein, 39 aa, Escherichia sp.; identical to SEQ ID NO: 18

SEQ ID NO: 57 - protein, 55 aa, Pseudomonas phage PP7
MSSTLCRWAVKALRCTRVYKEFIWKPLVALSYVTLYLLSSVFLSQLSYPIGSWAV

SEQ ID NO: 58 - protein, 35 aa, Acinetobacter phage AP205
MKKRTKALLPYAVFIILSFQLTLLTALFMYYHYTF

SEQ ID NO: 59 - protein, 38 aa, Alces alces faeces associated microvirus MP12 5423
MAKKIRNKARDRRIFTRTASRMHKANRTPRFMRGGIRL

SEQ ID NO: 60 - protein, 38 aa, Unknown
  Description of Unknown Gokushovirinae environmental samples sequence
MRRKKMSRGKSKKLFRRTAKRVHRKNLRARPMRGGIRM

SEQ ID NO: 61 - protein, 39 aa, Unknown
  Description of Unknown Gokushovirinae environmental samples sequence
MAKRHKIPQRASQHSFTRHAQKVHPKNVPRLPMRGGIRL

SEQ ID NO: 62 - protein, 37 aa, Unknown
  Description of Unknown uncultured bacterium sequence
MRKKMHKSLDKRVFNRTAKKSKKINVNPVVYRGGIRL

SEQ ID NO: 63 - protein, 38 aa, Marine gokushovirus; identical to SEQ ID NO: 24

SEQ ID NO: 64 - protein, 41 aa, Unknown
  Description of Unknown Richelia intracellularis HH01 sequence
MRPVKRSRVNKARSAGKFRKQVGKTKMANLRSNPMRGGWRL

SEQ ID NO: 65 - protein, 41 aa, Gokushovirinae Fen7875_21
MKPLKRKPVQKARSAAKFRRNVSTVKAANMAVKPMRGGWRF

SEQ ID NO: 66 - protein, 44 aa, Mycobacterium phage BabyRay
MTKRDIEYRKALGLNPSEPLPKIVGAVTRHGATLKRPRVTALAR

SEQ ID NO: 67 - protein, 38 aa, Unknown
  Description of Unknown Bdellovibrio phage phiMH2K sequence
MKRKPMSRKASQKTFKKNTGVQRMNHLNPRAMRGGIRL

SEQ ID NO: 68 - DNA, 120 bp, Escherichia sp.
atggctcgttctcgtcgtcgtatgtctaaacgttcttctcgtcgttcttttcgtaaatatgctaaaactcataaaaaaaattttaaagctcgttctatgcgtggaggaattcgtttataa

SEQ ID NO: 69 - DNA, 117 bp, Escherichia sp.
atggcgcgcagccgccgccgcatgagcaaacgcagcagccgccgcagctttcgcaaatatgcgaaaagccataaaaaaaactttaaagcgcgcagcatgcgcggcggcattcgcctg

SEQ ID NO: 70 - DNA, 168 bp, Pseudomonas phage PP7
ttgtcgtcaaccttgtgccgctgggccgttaaggccctgcggtgtacccgtgtgtataaggagtttatatggaaacccttagtagcgctcagttacgtgacgttgtatcttctgagctcggtcttcctgtcccaactcagctaccccatcgggagctgggcggtgtag

SEQ ID NO: 71 - DNA, 108 bp, Acinetobacter phage AP205
atgaagaaaaggacaaaagccttgcttccctatgcggttttcatcatactcagctttcaactaacattgttgactgccttgtttatgtattaccattataccttttag

SEQ ID NO: 72 - DNA, 117 bp, Alces alces faeces associated microvirus MP12 5423
atggcaaagaaaattagaaacaaagcacgtgatagacgtatcttcacaagaacagcttcacgcatgcacaaggcaaaccgcacaccaagatttatgagaggcggtattaggttatga

SEQ ID NO: 73 - DNA, 117 bp, Unknown
  Description of Unknown Gokushovirinae environmental samples sequence
atgcgtcgtaaaaaaatgtcacgcggtaaatcaaaaaaactctttcgccgaacagcaaaacgcgttcatcgaaaaaacctacgagctcgcccaatgcgtggcggcatacgcatgtag

SEQ ID NO: 74 - DNA, 120 bp, Unknown
  Description of Unknown Gokushovirinae environmental samples sequence
atggcgaagcgacacaaaatcccgcaacgcgcgtcacaacattccttcacgcgccatgcgcaaaaggtccaccctaagaacgttccccgcctgccaatgcgaggcggtatccgtctctaa

SEQ ID NO: 75 - DNA, 114 bp, Unknown
  Description of Unknown uncultured bacterium sequence
atgcgtaaaaaaatgcacaaatcattagacaagcgagtgtttaaccgcactgcaaaaaaatcaaaaaaaataaatgttaatcctgtagtttatcgtggaggtattagattatga

SEQ ID NO: 76 - DNA, 117 bp, Marine gokushovirus; identical to SEQ ID NO: 50

SEQ ID NO: 77 - DNA, 126 bp, Unknown
  Description of Unknown Richelia intracellularis HH01 sequence
atgcgtccagttaaaagatcaagagtaaataaggcccgatctgcaggcaagtttcgtaagcaggtcggtaaaacaaagatggcaaatctgcgtagtaatccgatgcgcggcggatggcggctgtga

SEQ ID NO: 78 - DNA, 126 bp, Gokushovirinae Fen7875_21
atgaagccattgaagcgtaagccggttcagaaggcgcggtcagcagccaagttccgtcgaaatgtgtctaccgttaaggctgccaatatggcggtgaagccgatgcgcggcggttggcggttctga

SEQ ID NO: 79 - DNA, 135 bp, Mycobacterium phage BabyRay
atgaccaagagagacatcgagtaccggaaagctttggggctcaacccatctgagccgctcccgaagattgtgggtgccgtcacccgccacggggccactctgaaacgcccacgggtcaccgcactggcccgatag

SEQ ID NO: 80 - DNA, 117 bp, Unknown
  Description of Unknown Bdellovibrio phage phiMH2K sequence
atgaaaagaaaaccaatgagccgcaaggcctctcaaaaaaccttcaaaaagaacacaggcgttcaacgcatgaaccatctcaacccacgcgccatgcgtggtggcattagactataa

(19) United States
(12) Patent Application Publication
(10) Pub. No.: US 2021/0330738 A1
(43) Pub. Date: Oct. 28, 2021

(54) ANTIMICROBIAL, BACTERIOPHAGE-DERIVED POLYPEPTIDES AND THEIR USE AGAINST GRAM-NEGATIVE BACTERIA

(71) Applicant: CONTRAFECT CORPORATION, Yonkers, NY (US)

(72) Inventor: Raymond SCHUCH, Mountain Lakes, NJ (US)

(21) Appl. No.: 17/041,638

(22) PCT Filed: Mar. 29, 2019
(86) PCT No.: PCT/US19/24854

§ 371 Date: Sep. 25, 2020

Related U.S. Application Data
(60) Provisional application No. 62/650,235, filed on Mar. 29, 2018.

(51) Int. Cl. A61K 38/16; C07K 14/005; A61K 31/7052; A61K 31/427; A61K 31/665; A61K 31/546; A61K 31/496; A61K 31/5383; A61K 31/407; A61K 31/7036; A61K 31/43; A61K 38/12; A61P 31/04

(52) U.S. Cl. CPC A61K 38/162; C07K 14/005; A61K 31/7052; A61K 31/427; A61K 31/665; A61K 31/546; A61K 48/00; A61K 31/5383; A61K 31/407; A61K 31/7036; A61K 31/43; A61K 38/12; A61P 31/04; A61K 31/496

(57) ABSTRACT

Disclosed herein are pharmaceutical compositions comprising an effective amount of an isolated Chp peptide having an amino acid sequence selected from the group consisting of SEQ ID NOs. 1-4, 6-26 and 54-66, or a modified Chp peptide having about 80% sequence identity therewith, wherein the modified Chp peptide inhibits growth, reduces the population, or kills at least one species of Gram-negative bacteria; and a pharmaceutically acceptable carrier. Further disclosed herein are isolated Chp peptides, as well as vectors comprising a nucleic acid molecule that encode the Chp peptides and host cells comprising a vector. Also disclosed herein are methods of inhibiting the growth, reducing the population, or killing of at least one species of Gram-negative bacteria and methods of treating a bacterial infection in a subject.

### CROSS-REFERENCE TO RELATED APPLICATIONS

This application claims the benefit of, and relies on the filing date of, U.S. provisional patent application No. 62/650,235, filed 29 Mar. 2018, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Mar. 28, 2019, is named 0341_0002-PCT_SL.txt and is 28,097 bytes in size.

### FIELD OF THE DISCLOSURE

The present disclosure relates to the field of antimicrobial agents and more specifically to phage-derived antimicrobial amurin peptides that infect Gram-negative bacteria and the use of these peptides in killing Gram-negative bacteria and combatting bacterial infection and contamination.

### BACKGROUND OF THE DISCLOSURE

Gram-negative bacteria, in particular, members of the genus Pseudomonas and the emerging multi-drug resistant pathogen Acinetobacter baumannii, are an important cause of serious and potentially life-threatening invasive infections. Pseudomonas infection presents a major problem in burn wounds, chronic wounds, chronic obstructive pulmonary disorder (COPD), cystic fibrosis, surface growth on implanted biomaterials, and within hospital surface and water supplies where it poses a host of threats to vulnerable patients.

Once established in a patient, P. aeruginosa can be especially difficult to treat. The genome encodes a host of resistance genes, including multidrug efflux pumps and enzymes conferring resistance to beta-lactam and aminoglycoside antibiotics, making therapy against this Gram-negative pathogen particularly challenging due to the lack of novel antimicrobial therapeutics. This challenge is compounded by the ability of P. aeruginosa to grow in a biofilm, which may enhance its ability to cause infections by protecting bacteria from host defenses and chemotherapy.

In the healthcare setting, the incidence of drug-resistant strains of Pseudomonas aeruginosa is increasing. In an observational study of health care-associated bloodstream infections (BSIs) in community hospitals, P. aeruginosa was one of the top four Multiple Drug Resistant (MDR) pathogens, contributing to an overall hospital mortality of 18%. Additionally, outbreaks of MDR P. aeruginosa are well-documented. Poor outcomes are associated with MDR strains of P. aeruginosa that frequently require treatment with drugs of last resort, such as colistin.

Other drug-resistant bacteria that have been identified as significant threats by the World Health Organization (WHO) and Centers for Disease Control (CDC) include the following Gram-negative bacteria: Acinetobacter baumannii, Pseudomonas aeruginosa, Enterobacteriaceae (including Escherichia coli, Klebsiella pneumoniae, and Enterobacter cloacae), Salmonella species, Neisseria gonorrhoeae, and Shigella species (Tillotson G. 2018. A crucial list of pathogens. Lancet Infect Dis 18:234-236).

To address the need for new antimicrobials with novel mechanisms, researchers are investigating a variety of drugs and biologics. One such class of antimicrobial agents includes lysins. Lysins are cell wall peptidoglycan hydrolases, which act as “molecular scissors” to degrade the peptidoglycan meshwork responsible for maintaining cell shape and for withstanding internal osmotic pressure. Degradation of peptidoglycan results in osmotic lysis. However, certain lysins have not been effective against Gram-negative bacteria, at least in part, due to the presence of an outer membrane (OM), which is absent in Gram-positive bacteria and which limits access to subjacent peptidoglycan. Modified lysins (“artilysins”) have also been developed. These agents, which contain lysins fused to specific a-helical domains with polycationic, amphipathic, and hydrophobic features, are capable of translocating across the OM. However, certain artilysins exhibit low in vivo activity. This may be caused by constituents of human serum and specifically by physiologic salt and divalent cations. These constituents compete for lipopolysaccharide binding sites and may interfere with the a-helical translocation domains of lysins, thereby restricting activity in blood and limiting the effectiveness of certain lysins and artilysins for treating invasive infections. A similar lack of activity in blood has been reported for multiple different outer membrane-penetrating and destabilizing antimicrobial peptides.

In addition to lysins and artilysins, other phage-encoded host lysis systems have been identified, including “amurins” (Chamakura KR et al., 2017. Mutational analysis of the MS2 lysis protein L. Microbiology 163:961-969). The term amurin describes a limited set of nonmuralytic (not “wall-destroying,” i.e., not based on peptidoglycan hydrolysis of the cell wall) lysis activities from both ssDNA and ssRNA phages (Microviridae and Leviviridae, respectively). For example, the protein E amurin of phage φX174 (Family Microviridae, genus Microvirus) is a 91 amino acid membrane protein that causes lysis by inhibiting the bacterial translocase MraY, an essential membrane-embedded enzyme that catalyzes the formation of the murein precursor, Lipid I (Zheng Y et al., 2009. Purification and functional characterization of phiX174 lysis protein E. Biochemistry 48:4999-5006). Additionally, the A2 capsid protein of phage Qβ (Family Leviviridae, genus Allolevivirus) is a 420-amino acid structural protein (and amurin) that causes lysis by interfering with MurA activity and dysregulating the process of peptidoglycan biosynthesis (Gorzelnik K V et al., 2016. Proc Natl Acad Sci U.S.A. 113:11519-11524). Other non-limiting examples include the LysM amurin of phage M, which is a specific inhibitor of MurJ, the lipid II flippase of E. coli, and the protein L amurin of phage MS2 (Family Levivirdae, genus Levivirus), which is a 75 amino acid integral membrane protein and causes lysis in a manner requiring the activity of host chaperone DnaJ (Chamakura K R et al., 2017. J Bacteriol 199). A putative domain structure for the L-like amurins has been assigned and includes an internal leucylserine dipeptide immediately preceded by a stretch of 10-17 hydrophobic residues. These amurins are integral membrane proteins and have not been purified and used like lysins. Further, their targets are in the cytoplasm. They have not been tested as lytic agents. Some amurins have been described in detail, for example in PCT Published Application No. WO 2001/009382, but at best they constitute a basis for development of therapeutics and have not been developed into antibacterial therapeutics.

Although recent publications have described lysins/artilysins and other host lysis systems (e.g., amurins) that may be used against Gram-negative bacteria with varying levels of efficacy in vivo, there remains a need for additional antibacterial compounds that target MDR P. aeruginosa and other Gram-negative bacteria for the treatment of invasive infections, and especially antibacterial compounds that are highly soluble, remain active in vivo in the presence of serum, and/or do not have hemolytic activity.

### BRIEF DESCRIPTION OF THE DRAWINGS

FIG. 1A are three-dimensional models predicted by I-Tasser for structures of Chlamydia phage peptide (Chp) family members Chp1, Chp2, Chp4, Chp5, Chp6, Chp7, Ecp1, Ecp2, and Osp 1. The human innate immune effector peptide LL-37 is included for comparison. Alpha helical structures are evident, and the top terminal is generally the N-terminal.

FIG. 1B shows the consensus secondary structure predictions for Chp2 (SEQ ID NO: 2) using JPRED4. The alpha-helices are indicated by the thick striped bar.

FIG. 1C shows the consensus secondary structure predictions for Chp4 (SEQ ID NO: 4) using JPRED4. The alpha-helices are indicated by the thick striped bar

FIG. 2A is the rooted (UPGMA clustering method) phylogenetic tree of certain Chp family members generated from a ClustalW alignment.

FIG. 2B is the unrooted (neighbor-joining clustering method) phylogenetic tree of certain Chp family members generated from a ClustalW alignment.

FIG. 3 is a series of photomicrographs showing microscopic analysis (×2000 magnification) of Pseudomonas aeruginosa strain 1292 treated for 15 minutes with Chp2 (10 μg/mL) or a buffer control (“untreated”) in 100% human serum. Samples were stained using the Live/Dead Cell Viability Kit (ThermoFisher) and examined by both differential interference contrast (DIC) and fluorescence microscopy. The photomicrographs show an absence of dead bacteria in the untreated row and a reduction of live bacteria in the treated row.

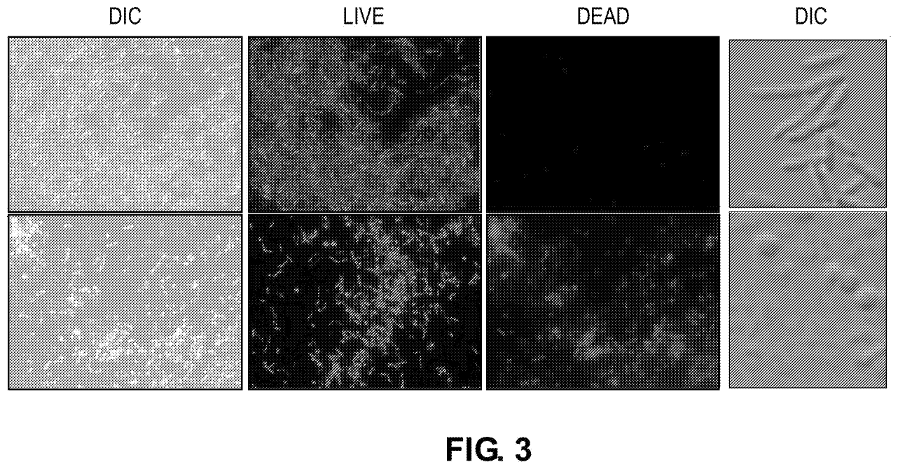

### SUMMARY OF THE DISCLOSURE

This application discloses a novel class of phage lytic agents that are derived, for example, from Microviridae genomic sequences and are distinct from other such agents, including known lysins/artilysins and amurins. The phage lytic agents disclosed herein are referred to as Chlamydia phage (Chp) peptides, also referred to as “amurin peptides” (a functional definition not implying sequence similarity with amurins). Disclosed herein are 40 Chp peptides that have been identified, constituting a family of specific bacteriolytic proteins. Several of the Chp peptides disclosed herein exhibit notable sequence similarities to each other but are distinct from other known peptides in the sequence databases. Despite the unique sequences of the Chp peptides, they are all predicted to adopt alpha-helical structures similar to some previously described antimicrobial peptides (AMPs) of vertebrate innate immune systems (E.F. Haney et al, 2017, In Hansen PR (ed), Antimicrobial Peptides: Methods and Protocols, Methods in Molecular Biology, vol. 1548) but with no sequence similarity to such AMPs. Consistent with an antibacterial function for the Chp class, disclosed herein is the potent and broad-spectrum bactericidal activity against Gram-negative pathogens for several different purified Chp peptides. Unlike the previously described amurins of Microviridae, which have cytoplasmic targets in the cell wall biosynthetic apparatus that may not be easily accessed by externally applied proteins, the Chp peptides disclosed herein can be used, in purified forms, to exert bactericidal activity “from without,” i.e., by acting on the outside of the cell wall. The Chp peptides identified here represent a novel class of antimicrobial agents having broad-spectrum activity against Gram-negative pathogens and the ability to persist in the presence of serum.

In one aspect, the present disclosure is directed to a pharmaceutical composition comprising a pharmaceutically acceptable carrier and an effective amount of (i) an isolated Chp peptide having an amino acid sequence selected from the group consisting of SEQ ID NOs. 1-4, 6-26 and 54-66 or active fragments thereof, or (ii) a modified Chp peptide having at least 80%, such as at least 85%, at least 90%, at least 92.5%, at least 95%, at least 98%, at least 99% sequence identity with at least one of SEQ ID NOs. 1-4, 6-26 and 54-66, wherein the modified Chp peptide inhibits the growth, reduces the population, and/or kills at least one species of Gram-negative bacteria, optionally in the presence of human serum. In certain embodiments, the at least one species of Gram-negative bacteria comprises Pseudomonas aeruginosa.

In another embodiment disclosed herein, the pharmaceutical composition comprises a pharmaceutically acceptable carrier and an effective amount of an isolated Chp peptide selected from the group consisting of peptides Chp1, Chp2, Chp3, Chp4, Chp6, Chp7, Chp8, Chp9, Chp10, Chp11, Chp12, CPAR39, Gkh1, Gkh2, Unp1, Ecp1, Tma1, Ecp2, Osp1, Unp2, Unp3, Gkh3, Unp5, Unp6, Spi1, Spi2, Ecp3, Ecp4, Lvp1, Lvp2, ALCES1, AVQ206, AVQ244, CDL907, AGT915, HH3930, Fen7875, and SBR77 or active fragments thereof.

In some embodiments, the Chp peptide is Chp2, Chp4, Chp6, Ecp1 or Ecp2.

In various embodiments of the disclosure, the pharmaceutical composition comprises a pharmaceutically acceptable carrier and an effective amount of (i) an isolated Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; and SEQ ID NO: 66 or active fragments thereof.

In certain embodiments, the pharmaceutical composition comprises a pharmaceutically acceptable carrier and an effective amount of (i) an isolated Chp peptide having an amino acid sequence selected from the group consisting of SEQ ID NO: 2; SEQ ID NO: 4; SEQ ID NO: 6, SEQ ID NO: 16; SEQ ID NO: 18; and SEQ ID NO: 54 or active fragments thereof.

In certain embodiments, the Chp peptide as disclosed herein or active fragments thereof contains at least one non-natural modification relative to the amino acid sequence of any one of SEQ ID NOs. 1-4, 6-26 and 54-66, and in certain embodiments, the non-natural modification is selected from the group consisting of substitution modification, such as a substitution of an amino acid; an N-terminal acetylation modification; and a C-terminal amidation modification. In certain embodiments, the modified Chp peptide comprises at least one amino acid substitution, insertion, or deletion relative to the amino acid sequence of any one of SEQ ID NOs. 1-4, 6-26 and 54-66, wherein the modified Chp peptide inhibits the growth, reduces the population, and/or kills at least one species of Gram-negative bacteria, optionally in the presence of human serum. In certain embodiments, the at least one species of Gram-negative bacteria comprises Pseudomonas aeruginosa. In certain embodiments, the at least one amino acid substitution is a conservative amino acid substitution. In certain embodiments, the modified Chp peptide comprising at least one amino acid substitution relative to the amino acid sequence of any one of SEQ ID NOs. 1-4, 6-26 and 54-66 is a cationic peptide having at least one alpha helix domain.

The pharmaceutical composition in some embodiments may be a solution, a suspension, an emulsion, an inhalable powder, an aerosol, or a spray. In some embodiments the pharmaceutical composition may also comprise one or more antibiotics suitable for the treatment of Gram-negative bacteria. Optionally, the peptide Chp1 is excluded such that the pharmaceutical composition does not comprise Chp1.

In certain embodiments, disclosed herein is a vector comprising a nucleic acid that encodes (i) a Chp peptide having an amino acid sequence selected from the group consisting of SEQ ID NOs. 1-4, 6-26 and 54-66 or active fragments thereof, or (ii) a Chp peptide having at least 80%, at least 85%, at least 90%, at least 92.5%, at least 95%, at least 98%, or at least 99% sequence identity with at least one of SEQ ID NOs. 1-4, 6-26 and 54-66, wherein the modified Chp peptide inhibits the growth, reduces the population, and/or kills at least one species of Gram-negative bacteria, optionally in the presence of human serum. In certain embodiments, the at least one species of Gram-negative bacteria comprises Pseudomonas aeruginosa.

Also disclosed herein are recombinant expression vectors comprising a nucleic acid encoding (i) a Chp peptide comprising an amino acid sequence selected from the group consisting of SEQ ID NOs. 1-4, 6-26 and 54-66 or active fragments thereof, or (ii) a modified Chp peptide having at least 80%, at least 85%, at least 90%, at least 92.5%, at least 95%, at least 98%, or at least 99% sequence identity with at least one of SEQ ID NOs. 1-4, 6-26 and 54-66, wherein the modified Chp peptide inhibits the growth, reduces the population, and/or kills at least one species of Gram-negative bacteria, optionally in the presence of human serum. In certain embodiments, the at least one species of Gram-negative bacteria comprises Pseudomonas aeruginosa. In certain embodiments, the nucleic acid is operatively linked to a heterologous promoter. In certain embodiments, the nucleic acid encodes a Chp peptide comprising an amino acid sequence selected from the group consisting of the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragments thereof, and in certain embodiments, the nucleic acid encodes a Chp peptide comprising an amino acid sequence selected from the group consisting of SEQ ID NO: 2; SEQ ID NO: 4; SEQ ID NO: 6; SEQ ID NO: 16; SEQ ID NO: 18; and SEQ ID NO: 54 or active fragments thereof.

Further embodiments disclosed herein include an isolated host cell comprising the foregoing vectors. In some embodiments, the nucleic acid sequence is a cDNA sequence.

In yet another aspect, the disclosure is directed to isolated, purified nucleic acid encoding a Chp peptide comprising an amino acid sequence selected from the group consisting of SEQ ID NOs. 1-26 and 54-66 or active fragments thereof. In certain embodiments, the nucleic acid encodes a Chp peptide comprising an amino acid sequence selected from the group consisting of SEQ ID NOs. 1-4, 6-26 and 54-66 or active fragments thereof. In an alternative embodiment, the isolated, purified DNA comprises a nucleotide sequence selected from the group consisting of SEQ ID NOs. 27-53 and 68-80, and in certain embodiments, the isolated, purified DNA comprises a nucleotide sequence selected from the group consisting of SEQ ID NOs. 27-30, 32-53, and 68-79. Optionally, the nucleic acid is cDNA. In certain embodiments, the nucleotide sequence contains at least one non-natural modification, such as a mutation (e.g., substitution, insertion, or deletion) or a nucleic acid sequence encoding an N-terminal modification or a C-terminal modification.

In other aspects, the present disclosure is directed to various methods/uses. One such use is a method for inhibiting the growth, reducing the population, and/or killing of at least one species of Gram-negative bacteria, the method comprising contacting the bacteria with a composition comprising an effective amount of (i) a Chp peptide comprising an amino acid sequence selected from the group consisting of SEQ ID NOs. 1-4, 6-26 and 54-66 or active fragments thereof, or (ii) a modified Chp peptide having at least 80%, such as at least 85%, at least 90%, at least 92.5%, at least 95%, at least 98%, or at least 99% sequence identity therewith, wherein the modified Chp peptide inhibits said growth, reduces said population, and/or kills said at least one species of Gram negative bacteria. In certain embodiments, the Chp peptide comprises an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragments thereof, and in certain embodiments, the Chp peptide comprises an amino acid sequence selected from the group consisting of SEQ ID NO: 2; SEQ ID NO: 4; SEQ ID NO: 6; SEQ ID NO: 16; SEQ ID NO: 18; and SEQ ID NO: 54 or active fragments thereof.

Also disclosed herein is a method for inhibiting the growth of, reducing the population of, and/or killing at least one species of Gram-negative bacteria, the method comprising contacting the bacteria with a composition comprising an effective amount of a Chp peptide selected from the group consisting of Chp1, Chp2, Chp3, Chp4, Chp6, Chp7, Chp8, Chp9, Chp10, Chp11, Chp12, CPAR39, Gkh1, Gkh2, Unp1, Ecp1, Tma1, Ecp2, Osp1, Unp2, Unp3, Gkh3, Unp5, Unp6, Spi1, Spi2, Ecp3, Ecp4, Lvp1, Lvp2, ALCES1, AVQ206, AVQ244, CDL907, AGT915, HH3930, Fen7875, and SBR77 or active fragments thereof, wherein the Chp peptide or active fragments thereof have the property of inhibiting the growth, reducing the population, and/or killing at least one species of Gram-negative bacteria.

In certain embodiments, the at least one species of Gram-negative bacteria is Pseudomonas aeruginosa, and in certain embodiments, the method further comprises killing at least one other species of Gram-negative bacteria in addition to Pseudomonas aeruginosa.

Also disclosed herein is a method for treating a bacterial infection caused by a Gram-negative bacteria, comprising administering a pharmaceutical composition as disclosed herein to a subject diagnosed with, at risk for, or exhibiting symptoms of a bacterial infection.

In any of the foregoing methods/uses, the Gram-negative bacteria may be at least one Gram-negative bacteria selected from the group consisting of Acinetobacter baumannii, Pseudomonas aeruginosa, Escherichia coli, Klebsiella pneumoniae, Enterobacter cloacae, Salmonella species, Neisseria gonorrhoeae, and Shigella species. In certain embodiments, the Gram-negative bacteria is Pseudomonas aeruginosa.

Also disclosed herein is a method for treating or preventing a topical or systemic pathogenic bacterial infection caused by a Gram-negative bacteria comprising administering a pharmaceutical composition as disclosed herein to a subject in need of treatment or prevention.

Further disclosed herein is a method for preventing or treating a bacterial infection comprising co-administering to a subject diagnosed with, at risk for, or exhibiting symptoms of a bacterial infection, a combination of a first amount of a pharmaceutical composition as disclosed herein and a second amount of an antibiotic suitable for the treatment of Gram-negative bacterial infection, wherein the first and the second amounts together are effective for preventing or treating the Gram-negative bacterial infection.

In some embodiments, the antibiotic suitable for the treatment of Gram-negative bacterial infection is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin. In certain embodiments, the antibiotic is selected from one or more of amikacin, azithromycin, aztreonam, ciprofloxacin, colistin, fosfomycin, gentamicin, imipenem, piperacillin, rifampicin, and tobramycin.

In yet another embodiment, there is disclosed a method for augmenting the efficacy of an antibiotic suitable for the treatment of Gram-negative bacterial infection, comprising co-administering the antibiotic in combination with a pharmaceutical composition as disclosed herein, wherein administration of the combination is more effective in inhibiting the growth, reducing the population, or killing the Gram-negative bacteria than administration of either the antibiotic or the pharmaceutical composition thereof individually.

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log10 (99.9%) or better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity, or DNA or protein synthesis in bacteria. Nonlimiting examples of antibiotics active against Gram-negative bacteria include cephalosporins, such as ceftriaxone-cefotaxime, ceftazidime, cefepime, cefoperazone, and ceftobiprole; fluoroquinolones such as ciprofloxacin and levofloxacin; aminoglycosides such as gentamicin, tobramycin, and amikacin; piperacillin, ticarcillin, imipenem, meropenem, doripenem, broad spectrum penicillins with or without beta-lactamase inhibitors, rifampicin, polymyxin B, and colistin.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, Gram-negative bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a Chp peptide and an antibiotic or any other antibacterial agent, in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of Chp peptides with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, a Chp peptide could be administered only initially within 24 hours of an additional antibiotic, and then the additional antibiotic use may continue without further administration of the Chp peptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive or Gram-negative bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-negative bacteria, whether such infection be systemic, topical or otherwise concentrated or confined to a particular organ or tissue.

“Polypeptide” is used herein interchangeably with the term “peptide” and refers to a polymer made from amino acid residues and generally having at least about 30 amino acid residues. The term includes not only polypeptides in isolated form, but also active fragments and derivatives thereof. The term “polypeptide” also encompasses fusion proteins or fusion polypeptides comprising a Chp peptide as described herein and maintaining, for example a lytic function. Depending on context, a polypeptide can be a naturally occurring polypeptide or a recombinant, engineered, or synthetically produced polypeptide. A particular Chp peptide can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining, e.g., lytic activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” may also refer to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” The open-ended expression “a polypeptide comprising” a certain structure includes larger molecules than the recited structure, such as fusion polypeptides.

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a Chp peptide or active fragment thereof and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more Chp peptides or active fragments thereof. These can be used to make a fusion polypeptide with lytic activity.

“Active fragment” refers to a portion of a polypeptide that retains one or more functions or biological activities of the isolated polypeptide from which the fragment was taken, for example bactericidal activity against one or more Gram-negative bacteria.

“Amphipathic peptide” refers to a peptide having both hydrophilic and hydrophobic functional groups. In certain embodiments, secondary structure may place hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides that are synthetically produced peptides composed of mostly positively charged amino acid residues, such as lysine (Lys) and/or arginine (Arg) residues. The amino acid residues that are not positively charged can be neutrally charged amino acid residues, negatively charged amino acid residues, and/or hydrophobic amino acid residues.

“Hydrophobic group” refers to a chemical group such as an amino acid side chain that has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of a bacteria in the subject and thereby controlling or reducing a bacterial infection in a subject or bacterial contamination of an organ, tissue, or environment. Thus “treatment” that reduces incidence may, for example, be effective to inhibit growth of at least one Gram-negative bacterium in a particular milieu, whether it be a subject or an environment. On the other hand, “treatment” of an already established infection refers to inhibiting the growth, reducing the population, killing, including eradicating, a Gram-negative bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitute examples of prevention.

“Contracted diseases” refers to diseases manifesting with clinical or subclinical symptoms, such as the detection of fever, sepsis, or bacteremia, as well as diseases that may be detected by growth of a bacterial pathogen (e.g., in culture) when symptoms associated with such pathology are not yet manifest.

The term “derivative” in the context of a peptide or polypeptide or active fragments thereof is intended to encompass, for example, a polypeptide modified to contain one or more chemical moieties other than an amino acid that do not substantially adversely impact or destroy the lytic activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to Chp peptides include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a Chp peptide or fused to a Chp peptide without interfering with normal functions of cellular proteins. In certain embodiments, a polynucleotide encoding a fluorescent protein may be inserted upstream or downstream of the Chp polynucleotide sequence. This will produce a fusion protein (e.g., Chp Peptide::GFP) that does not interfere with cellular function or function of a Chp peptide to which it is attached. Polyethylene glycol (PEG) conjugation to proteins has been used as a method for extending the circulating half-life of many pharmaceutical proteins. Thus, in the context of Chp peptide derivatives, the term “derivative” encompasses Chp peptides chemically modified by covalent attachment of one or more PEG molecules. It is anticipated that pegylated Chp peptides will exhibit prolonged circulation half-life compared to the unpegylated Chp peptides, while retaining biological and therapeutic activity.

“Percent amino acid sequence identity” refers to the percentage of amino acid residues in a candidate sequence that are identical with the amino acid residues in the reference polypeptide sequence, such as a specific Chp peptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to Chp peptides as well. Thus, the term “substantially identical” will encompass mutated, truncated, fused, or otherwise sequence-modified variants of isolated Chp polypeptides and peptides described herein, and active fragments thereof, as well as polypeptides with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide.

As used herein, two amino acid sequences are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides of the present disclosure are substantially homologous when one or more, such as up to 10%, up to 15%, or up to 20% of the amino acids of the polypeptide, such as the Chp peptides described herein, are substituted with a similar or conservative amino acid substitution, and wherein the resulting peptides have at least one activity (e.g., antibacterial effect) and/or bacterial specificities of the reference polypeptide, such as the Chp peptides disclosed herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Suitable” in the context of an antibiotic being suitable for use against certain bacteria refers to an antibiotic that was found to be effective against those bacteria even if resistance subsequently developed.

“Outer Membrane” or “OM” refers to a feature of Gram-negative bacteria. The outer membrane is comprised of a lipid bilayer with an internal leaflet of phospholipids and an external amphiphilic leaflet largely consisting of lipopolysaccharide (LPS). The LPS has three main sections: a hexa-acylated glucosamine-based phospholipid called lipid A, a polysaccharide core and an extended, external polysaccharide chain called 0-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the peptidoglycan forms a thin layer that is very sensitive to hydrolytic cleavage—unlike the peptidoglycan of Gram-negative bacteria which is 30-100 nanometers (nm) thick and consists of up to 40 layers, the peptidoglycan of Gram-negative bacteria is only 2-3 nm thick and consists of only 1-3 layers.

### Microviridae Phages

Members of the phage family Microviridae may be of particular interest as potential sources of anti-infective agents for several reasons. As disclosed herein, it has been found that a large subset of these phages, including those of the genus Chlamydiamicrovirus (Family Microvirus, subfamily Gokushovirinae), have no conserved amurin sequence and instead encode small, uncharacterized cationic peptides that appear to form the basis of a heretofore uncharacterized lytic system. Additionally, bacteriophages of the family Microviridae infect medically-relevant organisms, including members of the families Enterobacteriaceae, Pseudomonadaceae, and Chlamydiaceae (Doore S M et al, 2016. Virology 491:45-55.). They also lack amurins and instead, as disclosed herein, encode unique uncharacterized antimicrobial-like peptides (called amurin peptides) that have not been previously identified or had a function ascribed to them. It was reasoned that if the putative antimicrobial-like peptides act in a manner similar to previously described antimicrobial peptides (AMPs), they would then be predicted to enable “lysis from without” in a manner not possible with the amurins and their cytoplasmic targets.

Based on a bioinformatics analysis of all annotated Microviridae genomic sequences in GenBank (with a focus on phages that lack amurins), 40 novel and syntenic open reading frames were identified. They encode small cationic peptides with predicted alpha-helical structures similar to AMPs (but with amino acid sequences dissimilar to AMPs) from the innate immune systems of a variety of vertebrates. These peptides, collectively referred to as “Chp peptides” or “amurin peptides,” are primarily found in the Chlamydiamicrovirus genus and, to a lesser extent, in other related members of the subfamily Gokushovirinae. See, e.g., Tables 1 and 2 below. The Chp peptides from a range of Microviridae phages may exhibit 30-100% identity to each other and may have no or little homology with other peptides in the protein sequence database. See, e.g., Table 3 below. Based on the prediction that the Chp peptides possess AMP-like activities, the 39 different family members were synthesized (Chp2 and Chp3 being identical amino acid sequences) for analysis in different Aspartate Aminotransferase (AST) assays. Based on minimum inhibitory concentration (MIC) values of 0.25-4 μg/mL in the presence of human serum, several Chp peptides have demonstrated superior serum activity compared to a group of up to 17 known AMPs tested (including innate immune effectors and derivatives thereof). Furthermore, activity against a range of Gram-negative pathogens has been demonstrated, including several on the World Health Organization (WHO) and Centers for Disease Control (CDC) priority lists, including P. aeruginosa, E. coli, E. cloacae, K. pneumoniae, A. baumannii, and S. typhimurium.

For at least two of the potent Chp peptides, Chp2 and Chp4, the ability to synergize in vitro with a range of up to 11 antibiotics against P. aeruginosa, including antibiotics used in the clinical treatment of Gram-negative infections, has been demonstrated. Additionally, both Chp2 and Chp4 were shown to have potent anti-biofilm activities in the MBEC assay format (MBEC=0.25 μg/mL) and bactericidal activity in the time-kill assay format at concentrations down to 1 μg/mL or lower. See Example 5, below.

Overall, these findings are consistent both with a role for the Chp family members in the process of host cell lysis (in the context of the bacteriophage lifecycle) and with the use of purified Chp peptides or derivatives thereof as broad-spectrum antimicrobial agents to target Gram-negative pathogens. One major drawback with the use of previously described AMPs as a treatment for invasive infections concerns toxicity to erythrocytes and a generalized membranolytic activity (i.e., hemolysis) (Oddo A. et al., 2017. Hemolytic Activity of Antimicrobial Peptides. Methods Mol Biol 1548:427-435). Generally, this may be tested in vitro using a standardized assay for detecting the lysis of human red blood cells. Many of the Chp peptides disclosed herein exhibit no hemolytic activity against human red blood cells, in contrast to several AMPs described in the literature (as well as Triton X-100) to have hemolytic activity. In certain embodiments, the Chp peptides disclosed herein may only exhibit minimum hemolytic activity or no hemolytic activity against human red blood cells, as compared to AMPs. Another drawback of AMPs described in the literature concerns a loss of activity in the presence of human blood matrices and physiological salt concentrations (Mohanram H. et al., 2016. Salt-resistant short antimicrobial peptides. Biopolymers 106:345-356); indeed, this effect of known AMPs can be observed in Table 6, below. The data provided herein demonstrate that certain Chp peptides are active in the presence of either human serum or plasma and/or active in growth media, such as Mueller Hinton broth and Casamino Acid medium, containing physiological salt concentrations. Although not wishing to be bound by theory, it is believed that the differences observed in activities of the Chp peptides and AMP peptides (in the literature) may be attributed to the distinct sources of the two types of agents, where the Chp peptides are from phage and the AMPs are based largely on innate immune effectors of vertebrate immune systems. The high activity of Chp peptides, the activity of Chp peptides in blood matrices, and/or the absence of hemolytic activity make them suitable for use in treating invasive diseases. For example, in certain embodiments, the Chp peptides may be active in nanomolar quantities.

In summary, while pathogen-specific targeted lysin therapeutics have the ability to serve as tailored therapy for serious mono-microbial infections caused by known MDR pathogens, there is still an unmet medical need for agents to address serious and life-threatening infections caused by polymicrobial resistant Gram-negative infections (e.g., certain intra-abdominal infections, as well as serious burn, surgical, and other wound infections). The Chp peptides disclosed herein help to meet this need because they have been shown here to exhibit potent activity against all major ESKAPE pathogens (Enterococcus faecium, Staphylococcus aureus, Klebsiella pneumoniae, Acinetobacter baumannii, Pseudomonas aeruginosa, and Enterobacter) commonly associated with MDR, and they are expected to be active against many Gram-negative bacteria. The Chp peptides disclosed herein may be active at high nanomolar concentrations, comparable to those of active lysins. The Chp peptides disclosed herein may also be responsible for highly potent, rapid, bacteriolytic effects, the ability to clear biofilms, synergy with conventional antibiotics, and synergy with each other, such as synergy between two or more Chp peptides.

Although the Chp peptides of the present disclosure need not be modified by the addition of antimicrobial peptides, in certain embodiments, the Chp peptides disclosed herein may be incorporated into a fusion protein. For example, a fusion protein may comprise a Chp peptide as disclosed herein and a lysin, such as a lysin active against Gram-negative bacteria. In certain embodiments, the Chp peptide may be added to the N-terminus or the C-terminus of a lysin with or without a linker sequence. It is contemplated that fusion polypeptides containing more than one bacteriolytic segment may contribute positively to the bacteriolytic activity of the parent lysin and/or the parent Chp peptide.

### Polypeptides

As demonstrated and explained herein, the Chp peptides described in this section, including wild-type Chp peptides, modified Chp peptides and derivatives or active fragments thereof, can be used in the pharmaceutical compositions and methods described herein.

In some embodiments, the Chp peptide is selected from at least one of Chp1 (SEQ ID NO: 1), Chp2 (SEQ ID NO: 2), CPAR39 (SEQ ID NO: 3), Chp3 (SEQ ID NO: 54); Chp4 (SEQ ID NO: 4), Chp6 (SEQ ID NO: 6), Chp? (SEQ ID NO: 7), Chp8 (SEQ ID NO: 8), Chp9 (SEQ ID NO: 9), Chp10 (SEQ ID NO: 10), Chp11 (SEQ ID NO: 11), Chp12 (SEQ ID NO: 12), Gkh1 (SEQ ID NO: 13), Gkh2 (SEQ ID NO: 14), Unp1 (SEQ ID NO: 15), Ecp1 (SEQ ID NO: 16), Tma1 (SEQ ID NO: 17), Ecp2 (SEQ ID NO: 18), Osp1 (SEQ ID NO: 19), Unp2 (SEQ ID NO: 20), Unp3 (SEQ ID NO: 21), Gkh3 (SEQ ID NO: 22), Unp5 (SEQ ID NO: 23), Unp6 (SEQ ID NO: 24), Spi1 (SEQ ID NO: 25), Spi2 (SEQ ID NO: 26), Ecp3 (SEQ ID NO: 55), Ecp4 (SEQ ID NO: 56); Lvp1 (SEQ ID NO: 57), Lvp2 (SEQ ID NO: 58), ALCES1 (SEQ ID NO: 59), AVQ206 (SEQ ID NO: 60), AVQ244 (SEQ ID NO: 61), CDL907 (SEQ ID NO: 62), AGT915 (SEQ ID NO: 63), HH3930 (SEQ ID NO: 64), Fen7875 (SEQ ID NO: 65), SBR77 (SEQ ID NO: 66), and Bdp1 (SEQ ID NO: 67) or active fragments thereof having lytic activity.

The Chp peptide may be a modified Chp peptide or active fragment thereof. In certain embodiments, the Chp peptide or active fragment thereof contains at least one modification relative to at least one of SEQ ID NOs. 1-4, 6-26 and 54-66, such as at least one amino acid substitution, insertion or deletion. In certain embodiments, the modified Chp peptide comprises a polypeptide sequence having at least 80%, such as at least 85%, such as at least 90%, such as at least 92.5%, such as at least 95%, such as at least 98%, or such as at least 99% sequence identity with the amino acid sequence of at least one Chp peptide selected from the group consisting of SEQ ID NOs. 1-4, 6-26 and 54-66 or an active fragment thereof, wherein the modified Chp peptide inhibits the growth, reduces the population, and/or kills at least one species of Gram-negative bacteria, such as, P. aeruginosa and optionally at least one additional species of Gram-negative bacteria as described herein, optionally in the presence of human serum.

In some embodiments, the Chp peptide is selected from (i) at least one of Chp1 (SEQ ID NO: 1), Chp2 (SEQ ID NO: 2), CPAR39 (SEQ ID NO: 3), Chp3 (SEQ ID NO: 54); Chp4 (SEQ ID NO: 4), Chp6 (SEQ ID NO: 6), Chp? (SEQ ID NO: 7), Chp8 (SEQ ID NO: 8), Chp10 (SEQ ID NO: 10), Chp11 (SEQ ID NO: 11), Ecp1 (SEQ ID NO: 16), Ecp2 (SEQ ID NO: 18), Ecp3 (SEQ ID NO: 55), Ecp4 (SEQ ID NO: 56), Osp1 (SEQ ID NO: 19), Unp2 (SEQ ID NO: 20), Gkh3 (SEQ ID NO: 22), Unp5 (SEQ ID NO: 23), Unp6 (SEQ ID NO: 24), Spil (SEQ ID NO: 25), Lvp1 (SEQ ID NO: 57), ALCES1 (SEQ ID NO: 59), AVQ206 (SEQ ID NO: 60), CDL907 (SEQ ID NO: 62), AGT915 (SEQ ID NO: 63), and SBR77 (SEQ ID NO: 66), or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 1-4, 6-8, 10, 11, 16, 18, 19, 21-25, 54-57, 59, 60, 62, 63, and 66, wherein the modified Chp peptide inhibits the growth, reduces the population, and/or kills Pseudomonas aeruginosa and at least additional one species of Gram-negative bacteria, optionally in the presence of human serum.

In some embodiments, the Chp peptide is selected from (i) at least one of Chp2 (SEQ ID NO: 2), Chp3 (SEQ ID NO: 54), Chp4 (SEQ ID NO: 4), Chp6 (SEQ ID NO: 6), Ecp1 (SEQ ID NO: 16), and Ecp2 (SEQ ID NO: 18), or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 2, 4, 6, 16, and 18, wherein the modified Chp peptide inhibits the growth, reduces the population, and/or kills at least one species of Gram-negative bacteria, such as, Pseudomonas aeruginosa and at least additional one species of Gram-negative bacteria, optionally in the presence of human serum.

In certain embodiments, the Chp peptide is selected from (i) at least one Chp peptide having an amino acid sequence selected from the group consisting of SEQ ID NO: 2; SEQ ID NO: 4; and SEQ ID NO: 6 or active fragments thereof, or (ii) a modified Chp peptide having at least 92.5% sequence identity with at least one of SEQ ID NOs. 2, 4, and 6, wherein the modified Chp peptide inhibits the growth, reduces the population, and/or kills at least one species of Gram-negative bacteria, such as, Pseudomonas aeruginosa and at least additional one species of Gram-negative bacteria, optionally in the presence of human serum.

In some embodiment, the Chp peptide of the present disclosure is a derivative of one of the reference Chp peptides that has been chemically modified. A chemical modification includes but is not limited to, adding chemical moieties, creating new bonds, and removing chemical moieties. Chemical modifications can occur anywhere in a Chp peptide, including the amino acid side chains, as well as the amino or carboxyl termini. For example, in certain embodiments, the Chp peptide comprises an N-terminal acetylation modification. In certain embodiments, the Chp peptide or active fragment thereof comprises a C-terminal amidation modification. Such modifications can be present at more than one site in a Chp peptide.

Furthermore, one or more side groups, or terminal groups of a Chp peptide or active fragment thereof may be protected by protective groups known to the person ordinarily-skilled in the art.

In some embodiments, the Chp peptides or active fragments thereof are conjugated to a duration enhancing moiety. In some embodiments, the duration enhancing moiety is polyethylene glycol. Polyethylene glycol (“PEG”) has been used to obtain therapeutic polypeptides of enhanced duration (Zalipsky, S., Bioconjugate Chemistry, 6:150-165 (1995); Mehvar, R., J. Pharm. Pharmaceut. Sci., 3:125-136 (2000), which is herein incorporated by reference in its entirety). The PEG backbone, (CH2CH2-0-)n, wherein n is a number of repeating monomers, is flexible and amphiphilic. When attached to another chemical entity, such as a Chp peptide or active fragment thereof, PEG polymer chains can protect such polypeptides from immune response and other clearance mechanisms. As a result, pegylation can lead to improved efficacy and safety by optimizing pharmacokinetics, increasing bioavailability, and decreasing immunogenicity and dosing amount and/or frequency.

### Polynucleotides

Chp Peptides and Active Fragments Thereof

In one aspect, the present disclosure is directed to an isolated polynucleotide comprising a nucleic acid molecule encoding a Chp peptide or active fragments thereof having lytic activity. As used herein “lytic activity” encompasses the ability of a Chp peptide to kill bacteria, reduce the population of bacteria or inhibit bacterial growth e.g., by penetrating the outer membrane of a Gram-negative bacteria (e.g., P. aeruginosa) in the presence or absence of human serum. Lytic activity also encompasses the ability to remove or reduce a biofilm and/or the ability to reduce the minimum inhibitory concentration (MIC) of an antibiotic in the presence and/or absence of human serum.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; and SEQ ID NO: 66 or active fragments thereof.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 14; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 62; SEQ ID NO: 63; SEQ ID NO: 64; SEQ ID NO: 65; and SEQ ID NO: 66 or active fragments thereof.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragment thereof, and in certain embodiments, the nucleic acid encodes a Chp peptide having an amino acid sequence selected from the group consisting of SEQ ID NO: 2; SEQ ID NO: 4; SEQ ID NO: 6, SEQ ID NO: 16; SEQ ID NO: 18; and SEQ ID NO: 54 or active fragments thereof.

In some embodiments, the Chp peptides disclosed herein and active fragments thereof are capable of penetrating the outer membrane of Gram-negative bacteria. Without being limited by theory, after penetration of the outer membrane, the Chp peptides or active fragments thereof can degrade peptidoglycan, a major structural component of the bacterial cell wall, resulting in cell lysis. In some embodiments, the Chp peptides or active fragments thereof disclosed herein contain positively charged (and amphipathic) N- and/or C-terminal a-helical domains that facilitate binding to the anionic outer membrane of a Gram-negative bacteria to effect translocation into the sub-adjacent peptidoglycan.

The ability of a Chp peptide or active fragment thereof to penetrate an outer membrane of a Gram-negative bacteria may be assessed by any method known in the art, such as described in WO 2017/049233, which is herein incorporated by reference in its entirety. For example, the Chp peptide or active fragment thereof may be incubated with Gram-negative bacteria and a hydrophobic compound. Most Gram-negative bacteria are strongly resistance to hydrophobic compounds, due to the presence of the outer membrane and, thus, do not allow the uptake of hydrophobic agents such as 1-N-phenylnaphthylamine (NPN), crystal violet, or 8-anilino-1-naphthalenesulfonic acid (ANS). NPN, for example, fluoresces strongly under hydrophobic conditions and weakly under aqueous conditions. Accordingly, NPN fluorescence can be used as a measurement of the outer membrane permeability.

More particularly, the ability of a Chp peptide or active fragment thereof to penetrate an outer wall may be assessed by incubating, e.g., NPN with a Gram-negative bacteria, e.g., P. aeruginosa strain PA01, in the presence of the Chp peptide or active fragment thereof to be tested for activity. A higher induction of fluorescence in comparison to the fluorescence emitted in the absence of a Chp peptide (negative control) indicates outer membrane penetration. In addition, fluorescence induction can be compared to that of established permeabilizing agents, such as EDTA (ethylene diamine tetraacetate) or an antibiotic such as an antibiotic of last resort used in the treatment of P. aeruginosa, i.e., Polymyxin B (PMB) to assess the level of outer membrane permeabilization.

In some embodiments, the Chp peptides disclosed herein or active fragments thereof exhibit lytic activity in the presence and/or absence of human serum. Suitable methods for assessing the activity of a Chp peptide or active fragment thereof in human serum are known in the art and described in the examples. Briefly, a MIC value (i.e., the minimum concentration of peptide sufficient to suppress at least 80% of the bacterial growth compared to control) may be determined for a Chp peptide or active fragment thereof and compared to, e.g., a compound inactive in human serum, e.g., T4 phage lysozyme or artilysin GN126. T4 phage lysozyme is commercially available, e.g. from Sigma-Aldrich, Inc. GN126 corresponds to Art-175, which is described in the literature and is obtained by fusing AMP SMAP-29 to GN lysin KZ144. See Briers et al. 2014, Antimicrob, Agents Chemother. 58:3774-3784, which is herein incorporated by reference in its entirety.

More particularly MIC values for a Chp peptide or active fragment thereof may be determined against e.g., the laboratory P. aeruginosa strain PA01, in e.g., Mueller-Hinton broth, Mueller-Hinton broth supplemented with human serum, CAA as described herein, which includes physiological salt concentrations, and CAA supplemented with human serum. The use of PA01 enables testing in the presence of elevated serum concentrations since unlike most clinical isolates, PA01 is insensitive to the antibacterial activity of human blood matrices.

In some embodiments, the Chp peptides disclosed herein or active fragments thereof are capable of reducing a biofilm. Methods for assessing the Minimal Biofilm Eradicating Concentration (MBEC) of a Chp peptide or active fragment thereof may be determined using a variation of the broth microdilution MIC method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method, fresh colonies of e.g., a P. aeruginosa strain, such as ATCC 17647, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; lnnovotech Inc.) and incubated e.g., 24 hours at 37° C. Biofilms are then washed and treated with e.g., a 2-fold dilution series of the lysin in TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum Chp peptide concentration required to remove at least 95% of the biofilm biomass assessed by crystal violet quantitation.

In some embodiments, the Chp peptides disclosed herein or active fragments thereof reduce the minimum inhibitory concentration (MIC) of an antibiotic in the presence and/or absence of human serum. Any known method to assess MIC may be used. In some embodiments, a checkerboard assay is used to determine the effect of a Chp peptide or active fragment thereof on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, P A, which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of Chp peptide or active fragment thereof diluted e.g., 2-fold along the vertical axis. The Chp peptide or active fragment thereof and antibiotic dilutions are then combined, so that each column has a constant amount of antibiotic and doubling dilutions of Chp peptide, while each row has a constant amount of Chp peptide and doubling dilutions of antibiotic. Each well thus has a unique combination of Chp peptide and antibiotic. Bacteria are added to the drug combinations at concentrations of 1×10⁵ GFU/ml in CAA, for example, with or without human serum. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣIFICmin) is used to determine the effect of the Chp peptide/antibiotic combination.

In some embodiments, the Chp peptides disclosed herein or active fragments thereof show low toxicity against erythrocytes. Any methodology known in the art may be used to assess the potential for hemolytic activity of the present Chp peptides or active fragments thereof.

In some embodiments, the isolated polynucleotides of the present disclosure comprise a nucleic acid molecule that encodes a modified Chp peptide, e.g., a Chp peptide containing one or more insertions, deletions and/or amino acid substitutions in comparison to a reference Chp peptide. Such reference Chp peptides include any one of SEQ ID NOs. 1-4, 6-26 and 54-66. In certain embodiments, the modified Chp peptide has at least 80%, at least 85%, at least 90%, at least 95%, at least 98%, or at least 99% sequence identity to a reference Chp polypeptide having the amino acid sequence selected from the group consisting of SEQ ID NOs. 1-4, 6-26 and 54-66.

The modified Chp peptides of the present disclosure are typically designed to retain an α-helix domain, the presence or absence of which can be readily determined using various software programs, such as Jpred4 (compio.dundee.ac.uk/jpred) and Helical Wheel (hael.net/helical.htm).

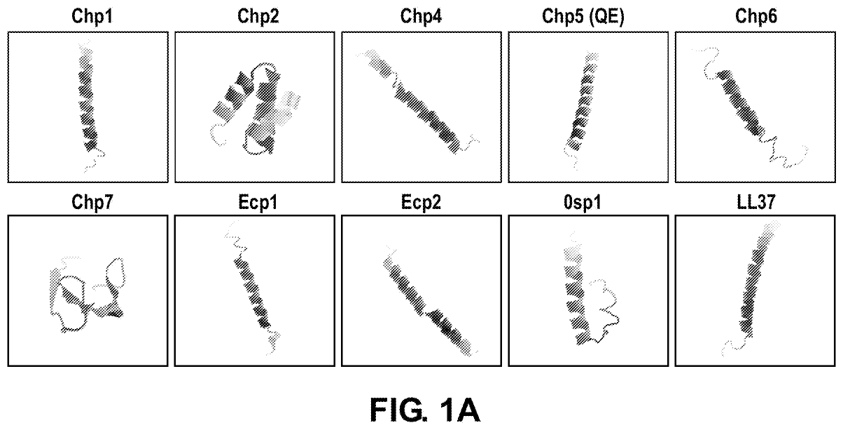

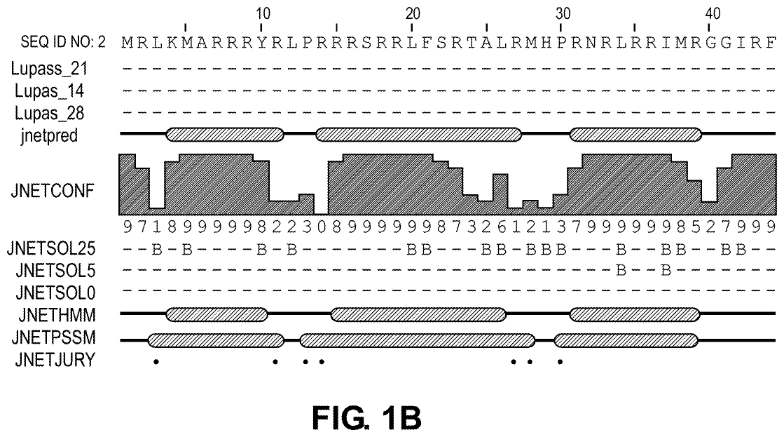

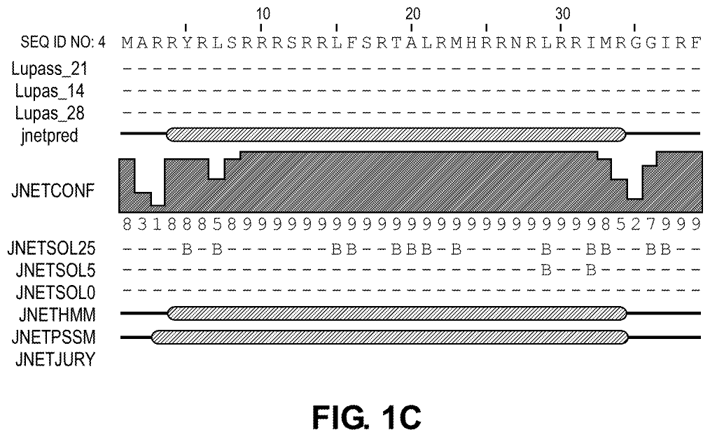

In some embodiments, the a-helix domain spans most of the molecule. See, e.g., Chp1 and Chp4 in FIG. 1. In some embodiments, the a-helix domain is interrupted (see, e.g., Chp2 in FIG. 1), and in some embodiments, the a-helix domain is truncated (see, e.g., Chp6 and Psp1 in FIG. 1). The a-helix domain of the Chp peptides of the present disclosure varies in size between about 3 and 32 amino acids, more typically between about 10 and 25 amino acid residues.

The modified Chp peptides of the present disclosure typically retain one or more functional or biological activities of the reference Chp peptide. In some embodiments, the modification improves the antibacterial activity of the Chp peptide. Typically, the modified Chp peptide has improved in vitro antibacterial activity (e.g., in buffer and/or media) in comparison to the reference Chp peptide. In other embodiments, the modified Chp peptide has improved in vivo antibacterial activity (e.g., in an animal infection model). In some embodiments, the modification improves the antibacterial activity of the Chp peptide in the absence and/or presence of human serum.

In some embodiments, Chp peptides disclosed herein or variants or active fragments thereof are capable of inhibiting the growth of, or reducing the population of, or killing P. aeruginosa and, optionally, at least one other species of Gram-negative bacteria in the absence or presence of, or in both the absence and presence of, human serum.

In some embodiments, the nucleic acid molecules of the present disclosure encode an active fragment of the Chp peptides or modified Chp peptides disclosed herein. The term “active fragment” refers to a portion of a full-length Chp peptide, which retains one or more biological activities of the reference peptide. Thus, an active fragment of a Chp peptide or modified Chp peptide, as used herein, inhibits the growth, or reduces the population, or kills P. aeruginosa and optionally at least one species of Gram-negative bacteria as described herein in the absence or presence of, or in both the absence and presence of, human serum. Typically, the active fragments retain an a-helix domain. In certain embodiments, the active fragment is a cationic peptide that retains an a-helix domain.

### Vectors and Host Cells

In another aspect, the present disclosure is directed to a vector comprising an isolated polynucleotide comprising a nucleic acid molecule encoding any of the Chp peptides or active fragments thereof disclosed herein or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; and SEQ ID NO: 66 or active fragments thereof.

In certain embodiments, the vector comprises a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 14; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 62; SEQ ID NO: 63; SEQ ID NO: 64; SEQ ID NO: 65; and SEQ ID NO: 66 or active fragments thereof.

In certain embodiments, the vector comprises a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragment thereof, and in certain embodiments, the vector comprises a nucleic acid molecule that encodes a Chp peptide having an amino acid sequence selected from the group consisting of SEQ ID NO: 2; SEQ ID NO: 4; SEQ ID NO: 6, SEQ ID NO: 16; SEQ ID NO: 18; and SEQ ID NO: 54 or active fragments thereof.

Generally, any system or vector suitable to maintain, propagate or express a polypeptide in a host may be used for expression of the Chp peptides disclosed herein or active fragments thereof. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the Chp peptides or active fragments thereof to provide convenient methods of isolation, e.g., c-myc, biotin, poly-His, etc. Kits for such expression systems are commercially available.

A wide variety of host/expression vector combinations may be employed in expressing the polynucleotide sequences encoding the Chp peptides disclosed herein or active fragments thereof. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the Chp peptides or active fragments thereof of the present disclosure. Suitable vectors include but are not limited to derivatives of SV40 and known bacterial plasmids, e.g., E. coli plasmids colE1, pCR1, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the Chp peptides or active fragments thereof of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the Chp peptides or active fragments thereof are operatively linked to a heterologous promoter or regulatory element.

In another aspect, the present disclosure is directed to a host cell comprising any of the vectors disclosed herein including the expression vectors comprising the polynucleotide sequences encoding the Chp peptides or active fragments thereof of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture. While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCS11O (Agilent Technologies, Inc.), JM109 (Promega, Inc.), LMG194 (ATCC), and BL21 (Thermo Fisher Scientific, Inc.).

There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in detail by Rosano, G. and Ceccarelli, E., Front Microbial., 5: 172 (2014).

Efficient expression of the present Chp peptides or active fragments thereof depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding Chp peptides or active fragments thereof of the present disclosure, one skilled in the art will be able to select the proper vectors, expression control sequences, and hosts without undue experimentation to accomplish the desired expression without departing from the scope of this disclosure.

Chp peptides or active fragments thereof of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also be employed for Chp peptide purification.

Alternatively, the vector system used for the production of Chp peptides or active fragments of the present disclosure may be a cell-free expression system. Various cell-free expression systems are commercially available, including, but are not limited to those available from Promega, LifeTechnologies, Clonetech, etc.

As indicated above, there is an array of choices when it comes to protein production and purification. Examples of suitable methods and strategies to be considered in protein production and purification are provided in WO 2017/049233, which is herein incorporated by reference in its entirety and further provided in Structural Genomics Consortium et al., Nat. Methods., 5(2): 135-146 (2008).

### Pharmaceutical Compositions

The compositions of the present disclosure can take the form of solutions, suspensions, emulsion, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

Administration of the compositions of the present disclosure or pharmaceutically acceptable forms thereof may be topical, i.e., the pharmaceutical composition may be applied directly where its action is desired (for example directly to a wound), or systemic. In turn, systemic administration can be enteral or oral, i.e., the composition may be given via the digestive tract, parenteral, i.e., the composition may be given by other routes than the digestive tract such as by injection or inhalation. Thus, the Chp peptides of the present disclosure and compositions comprising them can be administered to a subject orally, parenterally, by inhalation, topically, rectally, nasally, buccally, via an implanted reservoir, or by any other known method. The Chp peptides of the present disclosure or active fragments thereof can also be administered by means of sustained release dosage forms.

For oral administration, the Chp peptides of the present disclosure or active fragments thereof can be formulated into solid or liquid preparations, for example tablets, capsules, powders, solutions, suspensions, and dispersions. The composition can be formulated with excipients such as, e.g., lactose, sucrose, corn starch, gelatin, potato starch, alginic acid, and/or magnesium stearate.

For preparing solid compositions such as tablets and pills, a Chp peptide of the present disclosure or active fragments thereof may be mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The topical compositions of the present disclosure may further comprise a pharmaceutically or physiologically acceptable carrier, such as a dermatologically or an otically acceptable carrier. Such carriers, in the case of dermatologically acceptable carriers, may be compatible with skin, nails, mucous membranes, tissues, and/or hair, and can include any conventionally-used dermatological carrier meeting these requirements. In the case of otically acceptable carriers, the carrier may be compatible with all parts of the ear. Such carriers can be readily selected by one of ordinary skill in the art. Carriers for topical administration of the compositions of the present disclosure include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated, for example, in an oleaginous hydrocarbon base, an anhydrous absorption base, a water-in-oil absorption base, an oil-in-water water-removable base, and/or a water-soluble base. In formulating otic compositions, the active components of the present disclosure may be formulated, for example, in an aqueous polymeric suspension including such carriers as dextrans, polyethylene glycols, polyvinylpyrrolidone, polysaccharide gels, gellan gums such as Gelrite®, cellulosic polymers such as hydroxypropyl methylcellulose, and carboxy-containing polymers such as polymers or copolymers of acrylic acid, as well as other polymeric demulcents. The topical compositions according to the present disclosure may be in any form suitable for topical application, including aqueous, aqueous-alcoholic or oily solutions; lotion or serum dispersions; aqueous, anhydrous or oily gels; emulsions obtained by dispersion of a fatty phase in an aqueous phase (0/W or oil-in-water) or, conversely, (W/O or water-in-oil); microemulsions or alternatively microcapsules, microparticles or lipid vesicle dispersions of ionic and/or nonionic type; creams; lotions; gels; foams (which may use a pressurized canister, a suitable applicator, an emulsifier, and an inert propellant); essences; milks; suspensions; and patches. Topical compositions of the present disclosure may also contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. In a further aspect, the topical compositions disclosed herein may be administered in conjunction with devices such as transdermal patches, dressings, pads, wraps, matrices, and bandages capable of being adhered to or otherwise associated with the skin or other tissue of a subject, being capable of delivering a therapeutically effective amount of one or more Chp peptide or active fragment thereof as disclosed herein.

In one embodiment, the topical compositions of the present disclosure additionally comprise one or more components used to treat topical burns. Such components may include, but are not limited to, a propylene glycol hydrogel; a combination of a glycol, a cellulose derivative, and a water soluble aluminum salt; an antiseptic; an antibiotic; and a corticosteroid. Humectants such as solid or liquid wax esters; absorption promoters such as hydrophilic clays or starches; viscosity building agents; and skin-protecting agents may also be added. Topical formulations may be in the form of rinses such as mouthwash. See, e.g., WO2004/004650.

The compositions of the present disclosure may also be administered by injection of a therapeutic agent comprising the appropriate amount of a Chp peptide or active fragment thereof and a carrier. For example, the Chp peptide or active fragment thereof can be administered intramuscularly, intrathecally, subdermally, subcutaneously, or intravenously to treat infections by Gram-negative bacteria, such as those caused by P. aeruginosa. The carrier may be comprised of distilled water, a saline solution, albumin, a serum, or any combinations thereof. Additionally, pharmaceutical compositions of parenteral injections can comprise pharmaceutically acceptable aqueous or nonaqueous solutions of Chp peptides as disclosed herein or active fragments thereof in addition to one or more of the following: pH buffered solutions, adjuvants (e.g., preservatives, wetting agents, emulsifying agents, and dispersing agents), liposomal formulations, nanoparticles, dispersions, suspensions or emulsions, as well as sterile powders for reconstitution into sterile injectable solutions or dispersions just prior to use.

In cases where parenteral injection is the chosen mode of administration, an isotonic formulation may be used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application may be provided sterile and pyrogen free.

The diluent may further comprise one or more other excipient such as ethanol, propylene glycol, an oil, or a pharmaceutically acceptable emulsifier or surfactant.

In another embodiment, the compositions of the present disclosure are inhalable compositions. The inhalable compositions of the present disclosure can further comprise a pharmaceutically acceptable carrier. In one embodiment, the Chp peptides of the present disclosure or active fragments thereof may be formulated as a dry, inhalable powder. In specific embodiments, an inhalation solution comprising Chp peptides or active fragments thereof may further be formulated with a propellant for aerosol delivery. In certain embodiments, solutions may be nebulized.

A surfactant can be added to an inhalable pharmaceutical composition of the present disclosure in order to lower the surface and interfacial tension between the medicaments and the propellant. Where the medicaments, propellant and excipient are to form a suspension, a surfactant may or may not be used. Where the medicaments, propellant and excipient are to form a solution, a surfactant may or may not be used, depending, for example, on the solubility of the particular medicament and excipient. The surfactant may be any suitable, non-toxic compound which is non-reactive with the medicament and which reduces the surface tension between the medicament, the excipient and the propellant and/or acts as a valve lubricant.

Examples of suitable surfactants include, but are not limited to: oleic acid; sorbitan trioleate; cetyl pyridinium chloride; soya lecithin; polyoxyethylene (20) sorbitan monolaurate; polyoxyethylene (10) stearyl ether; polyoxyethylene (2) oleyl ether; polyoxypropylene-polyoxyethylene ethylene diamine block copolymers; polyoxyethylene (20) sorbitan monostearate; polyoxyethylene(20) sorbitan monooleate; polyoxypropylene-polyoxyethylene block copolymers; castor oil ethoxylate; and combinations thereof.

Examples of suitable propellants include, but are not limited to: dichlorodifluoromethane, trichlorofluoromethane, dichloro-tetrafluoroethane, and carbon dioxide.

Examples of suitable excipients for use in inhalable compositions include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the compositions of the present disclosure comprise nasal applications. Nasal applications include applications for direct use, such as nasal sprays, nasal drops, nasal ointments, nasal washes, nasal injections, nasal packings, bronchial sprays and inhalers, as well as applications for indirect use, such as throat lozenges and mouthwashes or gargles, or through the use of ointments applied to the nasal nares or the face, and any combination of these and similar methods of application.

In another embodiment, the pharmaceutical compositions of the present disclosure comprise a complementary agent, including one or more antimicrobial agents and/or one or more conventional antibiotics. In order to accelerate the treatment of the infection, or augment the antibacterial effect, the therapeutic agent containing a Chp peptide of the present disclosure or active fragment thereof may further include at least one complementary agent that can also potentiate the bactericidal activity of the peptide. The complementary agent may be one or more antibiotics used to treat Gram-negative bacteria. In one embodiment, the complementary agent is an antibiotic or antimicrobial agent used for the treatment of infections caused by P. aeruginosa.

The compositions of the present disclosure may be presented in unit dosage form and may be prepared by any methods well known in the art. The amount of active ingredients that can be combined with a carrier material to produce a single dosage form will vary depending, for example, upon the host being treated, the duration of exposure of the recipient to the infectious bacteria, the size and weight of the subject, and the particular mode of administration. The amount of active ingredients that can be combined with a carrier material to produce a single dosage form may, for example, be that amount of each compound which produces a therapeutic effect. In certain embodiments, out of one hundred percent, the total amount may range from about 1 percent to about ninety-nine percent of active ingredients, such as from about 5 percent to about 70 percent, or from about 10 percent to about 30 percent.

### Dosage and Administration

Dosages administered may depend on a number of factors such as the activity of infection being treated; the age, health and general physical condition of the subject to be treated; the activity of a particular Chp peptide or active fragment thereof; the nature and activity of the antibiotic if any with which a Chp peptide or active fragment thereof according to the present disclosure is being paired; and the combined effect of such pairing. In certain embodiments, effective amounts of the Chp peptide or active fragment thereof to be administered may fall within the range of about 1-50 mg/kg (or 1 to 50 mcg/ml). In certain embodiments, the Chp peptide or active fragment thereof may be administered 1-4 times daily for a period ranging from 1 to 14 days. The antibiotic if one is also used may be administered at standard dosing regimens or in lower amounts in view of any synergism. All such dosages and regimens, however, (whether of the Chp peptide or active fragment thereof or any antibiotic administered in conjunction therewith) are subject to optimization. Optimal dosages can be determined by performing in vitro and in vivo pilot efficacy experiments as is within the skill of the art but taking the present disclosure into account.

It is contemplated that the Chp peptides disclosed herein or active fragments thereof may provide a rapid bactericidal and, when used in sub-MIC amounts, may provide a bacteriostatic effect. It is further contemplated that the Chp peptides disclosed herein or active fragments thereof may be active against a range of antibiotic-resistant bacteria and may not be associated with evolving resistance. Based on the present disclosure, in a clinical setting, the present Chp peptides or active fragments thereof may be a potent alternative (or additive) for treating infections arising from drug- and multidrug-resistant bacteria alone or together with antibiotics (including antibiotics to which resistance has developed). It is believed that existing resistance mechanisms for Gram-negative bacteria do not affect sensitivity to the lytic activity of the present Chp peptides or active fragments thereof.

In some embodiments, time exposure to the Chp peptides disclosed herein or active fragments thereof may influence the desired concentration of active peptide units per ml. Carriers that are classified as “long” or “slow” release carriers (such as, for example, certain nasal sprays or lozenges) may possess or provide a lower concentration of peptide units per ml but over a longer period of time, whereas a “short” or “fast” release carrier (such as, for example, a gargle) may possess or provide a high concentration peptide units (mcg) per ml but over a shorter period of time. There are circumstances where it may be desirable to have a higher unit/ml dosage or a lower unit/ml dosage.

For the Chp peptides or active fragments thereof of the present disclosure, the therapeutically effective dose may be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration, in humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

A treatment regimen can entail administration daily (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration may be used. Dosage and frequency may vary depending on the patient. It will be understood by one of skill in the art that such guidelines will be adjusted for localized administration, e.g., intranasal, inhalation, rectal, etc., or for systemic administration, e.g., oral, rectal (e.g., via enema), intramuscular (i.m.), intraperitoneal (i.p.), intravenous (i.v.), subcutaneous (s.c.), transurethral, and the like.

### Methods

The Chp peptides and active fragments thereof of the present disclosure can be used in vivo, for example, to treat bacterial infections due to Gram-negative bacteria, such as P. aeruginosa, in a subject, as well as in vitro, for example to reduce the level of bacterial contamination on, for example, a surface, e.g., of a medical device.

For example, in some embodiments, the present Chp peptides or active fragments thereof may be used for the prevention, control, disruption, and treatment of bacterial biofilm formed by Gram-negative bacteria. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as the mucus plugs of the CF lung, contaminated catheters, contact lenses, etc (Sharma et al. Biologicals, 42(1):1-7 (2014), which is herein incorporated by reference in its entirety). Thus, in one embodiment, the Chp peptides or active fragments thereof of the present disclosure can be used for the prevention, control, disruption, and treatment of bacterial infections due to Gram-negative bacteria when the bacteria are protected by a bacterial biofilm.

In one aspect, the present disclosure is directed to a method of treating a bacterial infection caused by one or more additional of Gram-negative bacteria as described herein, comprising administering to a subject diagnosed with, at risk for, or exhibiting symptoms of a bacterial infection, a pharmaceutical composition as described herein described.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as gonococcal cervicitis and gonococcal urethritis; urinary tract infections; acute otitis media; sepsis including neonatal septisemia and catheter-related sepsis; and osteomyelitis. Infections caused by drug-resistant bacteria and multidrug-resistant bacteria are also contemplated.

Non-limiting examples of infections caused by Gram-negative bacteria, such as P. aeruginosa, include: A) Nosocomial infections: 1. Respiratory tract infections especially in cystic fibrosis patients and mechanically-ventilated patients; 2. Bacteremia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections; 2. Meningitis; 3. Folliculitis and infections of the ear canal caused by contaminated water; 4. Malignant otitis externa in the elderly and diabetics; 5. Osteomyelitis of the calcaneus in children; 6. Eye infections commonly associated with contaminated contact lens; 7. Skin infections such as nail infections in people whose hands are frequently exposed to water; 8. Gastrointestinal tract infections; and 9. Musculoskeletal system infections.

The one or more species of Gram-negative bacteria of the present methods may include any of the species of Gram-negative bacteria as described herein. Typically, the additional species of Gram-negative bacteria are selected from one or more of Acinetobacter baumannii, Acinetobacter haemolyticus, Actinobacillus actinomycetemcomitans, Aeromonas hydrophila, Bacteroides spp., such as, Bacteroides fragilis, Bacteroides theataioatamicron, Bacteroides distasonis, Bacteroides ovatus, Bacteroides vulgatus, Bartonella Quintana, Bordetella pertussis, Brucella spp., such as, Brucella melitensis, Burkholderia spp, such as, Burkholderia cepacia, Burkholderia pseudomallei, and Burkholderia mallei, Fusobacterium, Prevotella corporis, Prevotella intermedia, Prevotella endodontalis, Porphyromonas asaccharolytica, Campylobacter jejuni, Campylobacter fetus, Campylobacter coli, Chlamydia spp., such as Chlamydia pneumoniae and Chlamydia trachomatis, Citrobacter freundii, Citrobacter koseri, Coxiella burnetii, Edwarsiella spp., such as, Edwarsiella tarda, Eikenella corrodens, Enterobacter spp., such as, Enterobacter cloacae, Enterobacter aerogenes, and Enterobacter agglomerans, Escherichia coli, Francisella tularensis, Haemophilus influenzae, Haemophilus ducreyi, Helicobacter pylori, Kingella kingae, Klebsiella spp., such as, Klebsiella pneumoniae, Klebsiella oxytoca, Klebsiella rhinoscleromatis, and Klebsiella ozaenae, Legionella penumophila, Moraxella spp., such as, Moraxella catarrhalis, Morganella spp., such as, Morganella morganii, Neisseria gonorrhoeae, Neisseria meningitidis, P. aeruginosa, Pasteurella multocida, Plesiomonas shigelloides, Proteus mirabilis, Proteus vulgaris, Proteus penneri, Proteus myxofaciens, Providencia spp., such as, Providencia stuartii, Providencia rettgeri, Providencia alcalifaciens, Pseudomonas fluorescens, Salmonella typhi, Salmonella typhimurium, Salmonella paratyphi, Serratia spp., such as, Serratia marcescens, Shigella spp., such as, Shigella flexneri, Shigella boydii, Shigella sonnei, and Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydia pneumoniae, Chlamydia trachomatis, Ricketsia prowazekii, Coxiella burnetii, Ehrlichia chafeensis and/or Bartonella hensenae.

More typically, the at least one other species of Gram-negative bacteria is selected from one or more of Acinetobacter baumannii, Bordetella pertussis, Burkholderia cepacia, Burkholderia pseudomallei, Burkholderia mallei, Campylobacter jejuni, Campylobacter coli, Enterobacter cloacae, Enterobacter aerogenes, Escherichia coli, Francisella tularensis, Haemophilus influenzae, Haemophilus ducreyi, Helicobacter pylori, Klebsiella pneumoniae, Legionella pneumophila, Moraxella catarrhalis, Morganella morganii, Neisseria gonorrhoeae, Neisseria meningitidis, Pasteurella multocida, Proteus mirabilis, Proteus vulgaris, Salmonella typhi, Serratia marcescens, Shigella flexneri, Shigella boydii, Shigella sonnei, Shigella dysenteriae, Stenotrophomonas maltophilia, Vibrio cholerae, and/or Chlamydia pneumoniae.

Even more typically, the at least one other species of Gram-negative bacteria is selected from one or more of Salmonella typhimurium, Salmonella typhi, Shigella spp., Escherichia coli, Acinetobacter baumanii, Klebsiella pneumonia, Neisseria gonorrhoeae, Neisseria meningitides, Serratia spp. Proteus mirabilis, Morganella morganii, Providencia spp., Edwardsiella spp., Yersinia spp., Haemophilus influenza, Bartonella quintana, Brucella spp., Bordetella pertussis, Burkholderia spp., Moraxella spp., Francisella tularensis, Legionella pneumophila, Coxiella burnetii, Bacteroides spp., Enterobacter spp., and/or Chlamydia spp.

Yet even more typically, the at least one other species of Gram-negative bacteria is selected from one or more of Klebsiella spp., Enterobacter spp., Escherichia coli, Citrobacter freundii, Salmonella typhimurium, Yersinia pestis and/or Franciscella tularensis.

In some embodiments, infection with Gram-negative bacteria results in a localized infection, such as a topical bacterial infection, e.g., a skin wound. In other embodiments, the bacterial infection is a systemic pathogenic bacterial infection. Common Gram-negative pathogens and associated infections are listed in Table A of the present disclosure. These are meant to serve as examples of the bacterial infections that may be treated, mitigated or prevented with the present Chp peptides and active fragments thereof and are not intended to be limiting.

TABLE A
Medically relevant Gram-negative bacteria and associated diseases

| | |
|---|---|
| Salmonella typhimurium | Gastrointestinal (GI) infections- |
| | salmonellosis |
| Shigella spp. | shigellosis |
| Escherichia coli | Urinary tract infections (UTIs) |
| Acinetobacter baumanii | Wound infections |
| Pseudomonas aeruginosa | bloodstream infections and pneumonia |
| Klebsiella pneumoniae | UTIs, and bloodstream infections |
| Neisseria gonorrhoeae | Sexually transmitted disease (STD)- |
| | gonorrhea |
| Neisseria meningitides | Meningitis |
| Serratia spp. | Catheter contaminations, UTIs, and |
| | pneumonia |
| Proteus mirabilis | UTIs |
| Morganella spp. | UTIs |
| Providencia spp. | UTIs |
| Edwardsiella spp | UTIs |
| Salmonella typhi | GI infections - typhoid fever |
| Yersinia pestis | Bubonic and pneumonic plague |
| Yersinia enterocolitica | GI infections |
| Yersinia pseudotuberculosis | GI infections |
| Haemophilus influenza | Meningitis |
| Bartonella Quintana | Trench fever |
| Brucella spp. | Brucellosis |
| Bordetella pertussis | Respiratory - Whooping cough |
| Burkholderia spp. | Respiratory |
| Moraxella spp. | Respiratory |
| Francisella tularensis | Tularemia |
| Legionella pneumophila | Respiratory - Legionnaires' disease |
| Coxiella burnetii | Q fever |
| Bacteroides spp. | Abdominal infections |
| Enterobacter spp. | UTIs and respiratory |
| Chlamydia spp. | STDs, respiratory, and ocular |

In some embodiments, the Chp peptides and active fragments thereof of the present disclosure are used to treat a subject at risk for acquiring an infection due to Gram-negative bacterium. Subjects at risk for acquiring a Gram-negative bacterial infection include, for example, cystic fibrosis patients, neutropenic patients, patients with necrotising enterocolitis, burn victims, patients with wound infections, and, more generally, patients in a hospital setting, in particular surgical patients and patients being treated using an implantable medical device such as a catheter, for example a central venous catheter, a Hickman device, or electrophysiologic cardiac devices, for example pacemakers and implantable defibrillators. Other patient groups at risk for infection with Gram-negative bacteria include without limitation patients with implanted prostheses such a total joint replacement (for example total knee or hip replacement).

In another aspect, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a Chp peptide or active fragment thereof as described herein, and a second effective amount of an antibiotic suitable for the treatment of Gram-negative bacterial infection.

The Chp peptides and active fragments thereof of the present disclosure can be co-administered with standard care antibiotics or with antibiotics of last resort, individually or in various combinations as within the skill of the art. Traditional antibiotics used against P. aeruginosa are described in Table B. Antibiotics for other Gram-negative bacteria, such as Klebsiella spp., Enterobacter spp., Escherichia coli, Citrobacter freundii, Salmonella typhimurium, Yersinia pestis, and Franciscella tulerensis, are similar to that provided in Table B for P. aeruginosa.

TABLE B
Antibiotics used for the treatment of Pseudomonas aeruginosa

| Class | Agent |
|---|---|
| Penicillins | Ticarcillin-clavulanate |
| | Pi peracillin-tazobactam |
| Cephalosporins | Ceftazidime |
| | Cefepime |
| | Cefoperazone |
| Monobactams | Aztreonam |
| Flouroquinolones | Ci profloxacin |
| | Levofloxacin |
| Carbapemens | Imipenem |
| | Meropenem |
| | Doripenem |
| Aminogl ycosides | Gentamicin |
| | Tobramycin |
| | Amikacin |
| Polymixins | Colistin |
| | Polymixin B |

In more specific embodiments, the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B and colistin.

Combining the Chp peptides or active fragments thereof of the present disclosure with antibiotics provides an efficacious antibacterial regimen. In some embodiments, co-administration of Chp peptides or active fragments thereof of the present disclosure with one or more antibiotics may be carried out at reduced doses and amounts of either the Chp peptides or active fragments thereof or the antibiotic or both, and/or reduced frequency and/or duration of treatment with augmented bactericidal and bacteriostatic activity, reduced risk of antibiotic resistance and with reduced risk of deleterious neurological or renal side effects (such as those associated with colistin or polymyxin B use). Prior studies have shown that total cumulative colistin dose is associated with kidney damage, suggesting that decrease in dosage or shortening of treatment duration using the combination therapy with Chp peptides or active fragments thereof could decrease the incidence of nephrotoxicity (Spapen et al. Ann Intensive Care. 1: 14 (2011), which is herein incorporated by reference in its entirety). As used herein the term “reduced dose” refers to the dose of one active ingredient in the combination compared to monotherapy with the same active ingredient. In some embodiments, the dose of Chp peptides or active fragments thereof or the antibiotic in a combination may be suboptimal or even subthreshold compared to the respective monotherapy.

In some embodiments, the present disclosure provides a method of augmenting antibiotic activity of one or more antibiotics against Gram-negative bacteria compared to the activity of said antibiotics used alone by administering to a subject the Chp peptides or active fragments thereof disclosed herein together with an antibiotic of interest. The combination is effective against the bacteria and permits resistance against the antibiotic to be overcome and/or the antibiotic to be employed at lower doses, decreasing undesirable side effects, such as the nephrotoxic and neurotoxic effects of polymyxin B.

The Chp peptides or active fragments thereof optionally in combination with antibiotics of the present disclosure can be further combined with additional permeabilizing agents of the outer membrane of the Gram-negative bacteria, including, but not limited to metal chelators, such as e.g. EDTA, TRIS, lactic acid, lactoferrin, polymyxins, citric acid (Vaara M. Microbial Rev. 56(3):395-441 (1992), which is herein incorporated by reference in its entirety).

In yet another aspect, the present disclosure is directed to a method of inhibiting the growth, or reducing the population, or killing of at least one species of Gram-negative bacteria, the method comprising contacting the bacteria with a composition containing an effective amount of a Chp peptide or active fragment thereof as described herein, wherein the Chp peptide or active fragment thereof inhibits the growth, or reduces the population, or kills at least one species of Gram-negative bacteria.

In some embodiments, inhibiting the growth, or reducing the population, or killing at least one species of Gram-negative bacteria comprises contacting bacteria with the Chp peptides or active fragments as described herein, wherein the bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the Chp peptides or active fragments thereof described herein include but are not limited to tubing and other surface medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-negative bacteria.

### EXAMPLES

### Materials and Methods

Bacterial strains and growth conditions. The majority of studies disclosed herein were performed using a carbapenam-resistant P. aeruginosa clinical isolate CFS-1292 obtained from human blood at the Hospital for Special Surgery in New York (provided by Dr. Lars Westblade, Professor of Pathology and Laboratory Medicine), but commercially available antibiotic resistant isolates may also be used. All other isolates were obtained from either the American Type Culture Collection (“ATCC”), the d′Herelle collection (“HER”), BEI Resources (“HM”), or the Hospital for Special Surgery in New York (“HSS”). Isolates were cultured and tested in either lysogeny broth (LB; Sigma-Aldrich), casamino acid (CAA) media (5 g/L casamino acids, Ameresco/VWR; 5.2 mM K₂HPO₄, Sigma-Aldrich; 1 mM MgSO₄, Sigma-Aldrich), CAA supplemented with 100 mM NaCl, or CAA supplemented with 2.5% human serum (Type AB, male, pooled; Sigma-Aldrich). All antibiotics and protein reagents (e.g., T4 lysozyme) were obtained from Sigma-Aldrich unless otherwise indicated.

Bioinformatic studies. All proteins were identified in annotated GenBank database entries for all Microviridae and Leviviridae genomes. The accession number for each Chp group peptide is indicated in Tables 1 and 2 below. Blastp analyses were performed using the UniProt server, available at uniprot.org/blast/. Protein secondary structure predictions were performed using JPRED4, available at www.compbio.dundee.ac.uk/jpred/index, and I-Tasser, available at www.zhanglab.ccmb.med.umich.edu/I-TASSER/. Phylogenetic analyses were performed using ClustalW Multiple Sequence Alignment tools, available at www.genome.jp/tools-bin/clustalw. Predicted molecular weights and isoelectric points were determined using the ExPASy Resource Portal, available at web.expasy.org/compute_pi/.

Determination of Minimal Inhibitory Concentrations (MIC). MIC values were determined using a modification of the standard broth microdilution reference method defined by the Clinical and Laboratory Standards Institute (CLSI) (2015. Methods for Dilution Antimicrobial Susceptibility Tests for Bacteria That Grow Aerobically; Approved Standard-10th Edition. Clinical and Laboratory Standards Institute, Wayne, Pa.). The modification was based on the replacement of Mueller Hinton Broth, in some instances, with either CAA media (with and without NaCl) or CAA supplemented with 2.5% human serum. As used herein, MIC is the minimum concentration of peptide sufficient to suppress at least 80% of the bacterial growth compared to control.

Determination of Minimal Biofilm Eradicating Concentrations (MBEC). MBEC values were determined using a variation of the broth microdilution MIC method with modifications (Ceri H et al., 1999. J Clin Microbiol 37:1771-1776; and Schuch R et al., 2017. Antimicrob Agents Chemother 61). Fresh colonies of P. aeruginosa strain ATCC 17647 were suspended in PBS (0.5 McFarland units), diluted 1:100 in LB with 0.2% glucose, added as 0.15 ml aliquots to each well of a 96-well Calgary Biofilm Device (Innovotech), and incubated for 24 hours at 37° C. for the formation of biofilms on polycarbonate pegs. Biofilms were washed and treated with a 2-fold dilution series of each peptide in TSBg at 37° C. for 16 hours. After treatment, wells were washed, air-dried at 37° C., stained with 0.05% crystal violet for 10 minutes, and destained in 33% acetic acid. The OD600 of extracted crystal violet was determined. The MBEC value of each sample was determined as the minimum drug concentration required to remove >95% of biofilm biomass as assessed by crystal violet quantitation (in comparison to untreated controls). T4 phage lysozyme was used as a negative control and does not provide anti-biofilm activity.

Checkerboard assays. The checkerboard assay is based on a modification of the CLSI method for MIC determination by broth microdilution (CLSI 2015; and Moody J. 2010. Synergy testing: broth microdilution checkerboard and broth macrodilution methods, p 5.12.11-15.12.23. In Garcia LS (ed), Clinical Microbiology Procedures Handbook, vol 2). Checkerboards were constructed by first preparing columns of a 96-well polypropylene microtiter plate, in which each well had the same amount of antibiotic diluted 2-fold along the horizontal axis. In a separate plate, comparable rows were prepared in which each well had the same amount of peptide diluted 2-fold along the vertical axis. The peptide and antibiotic dilutions were then combined, so that each column had a constant amount of antibiotic and doubling dilutions of Gram-negative lysin, while each row had a constant amount of Gram-negative lysin and doubling dilutions of antibiotic. Each well thus had a unique combination of peptide and antibiotic. Bacteria were added to each well at a concentration of 1×105 CFU/mL in CAA with 2.5% human serum. The MIC of each agent, alone and in combination, was then recorded after 16 hours at 37° C. in ambient air. Summation fractional inhibitory concentration index (FICIs) were calculated for each drug and the minimum FICI was used to determine synergy. FICIs were calculated as follows: FICI=FIC A +FIC B, where FIC A is the MIC of each antibiotic in the combination/MIC of the each antibiotic alone, and FIC B is the MIC of each Gram-negative lysin in the combination/MIC of each Gram-negative lysin alone. The combination is considered synergistic when the FICI is <0.5, strongly additive when the FICI is >0.5 to <1, additive with the FICI is 1-<2, and antagonistic when the FICI is >2. Checkerboard assays were performed using P. aeruginosa strain CFS-1292 in CAA/HuS with combinations of either Chp2 or Chp4 against a range of 11 different antibiotics, including amikacin, azithromycin, aztreonam, ciprofloxacin, colistin, fosfomycin, gentamicin, imipenem, piperacillin, rifamipicin, and tobramycin. FICI values of <0.5 were observed for the majority of combinations, indicating the ability of Chp2 and Chp4 to synergize with a broad range of antibiotics (see Table 8 below). These findings suggest that the Chp peptides may provide potent antibacterial activity in the presence of antibiotics.

Assay of Gram Negative Lysin Hemolytic Activity. Hemolytic activity was measured as the amount of hemoglobin released by the lysis of human erythrocytes (Lv Y et al, 2014. PLoS One 9:e86364). Briefly, 3 ml of fresh human blood cells (hRBCs) obtained from pooled healthy donors (BioreclamationTVT) in a polycarbonate tube containing heparin were centrifuged at 1,000xg for 5 min at 4° C. The erythrocytes obtained were washed three times with phosphate-buffered saline (PBS) solution (pH 7.2) and resuspended in 30 ml PBS. A 50 μl volume of the erythrocyte solution was incubated with 50 μl of each Gram-negative lysin (in PBS) in a 2-fold dilution range (from 128 μg/mL to 0.25 μg/mL) for 1 h at 37° C. Intact erythrocytes were pelleted by centrifugation at 1,000×g for 5 min at 4° C., and the supernatant was transferred to a new 96-well plate. The release of hemoglobin was monitored by measuring the absorbance at an optical density (OD) of 570 nm. The minimal hemolytic concentration was determined as lowest peptide concentration exhibiting visual lysis (which corresponds to the minimal concentration resulting in an OD value ≥5% of the untreated control sample). Additional controls were used including hRBCs in PBS treated as above with either 0.1% Triton X-100 or each of a series of antimicrobial peptides with known hemolytic activity, including RR12, RR12polar and RR12hydrophobic (Mohanram H. et al, 2016. Biopolymers 106:345-356), and with little or no hemolytic activity, including RI18 (Lyu Y. et al., 2016. Sci Rep 6:27258) and RR22.

Time-Kill Assay of Gram Negative Lysin Activity. An overnight culture of P. aeruginosa strain CFS-1292 was diluted 1:00 into fresh CAA media with 2.5% human serum (CAA/HuS) and grown for 2.5 hours at 37° C. with agitation. Exponential phase bacteria were then diluted 1:100 into CAA/HuS and peptide was added at a final concentration of either 1 or 10 μg/mL. Control cultures were included with no peptide added (i.e., buffer control). Cultures were incubated at 37° C. with aeration and at 1 hr, 3 hr, and 24 hr time-points, samples were removed for quantitative plating on CAA agar plates.

Microscopy. Aliquots of P. aeruginosa strain CFS-1292, grown for 2.5 hours in LB, were washed with PBS and resuspended in either PBS or 100% human serum and treated for 15 minutes at room temperature with and without peptide Chp2 at a final concentration of 10 μg/mL. Sample subsets were stained using the Live/Dead Cell Viability Kit (ThermoFisher) according to the manufacturer's protocol and examined by differential interference contrast (DIC) microscopy and fluorescence microscopy.

### Example 1

### Identification of Chp Peptides

Having knowledge of certain poorly described bacteriophage (Chlamydiamicroviridae) that specifically infect and kill the Gram-negative bacteria Chlamydia, published genomes of these organisms were studied, initially looking to identify novel lysins, although no lysin-like sequences nor any sequences similar to previously described amurins were observed. Chlamydia do not utilize peptidoglycans (a known target of lysins) in their structures as abundantly as other bacteria, but rather Chlamydia generally only use peptidoglycans during division. Therefore, the question arose as to what the target of Chlamydia phage was. It was postulated that the mechanism by which Chlamydia phage invade their target may be different from the ones previously known, and their target may be different and focused on lipopolysaccharide (LPS), a main constituent of the outer membrane of Gram-negative bacteria and an obstacle to penetration by lysins of the outer membrane.

The published genomes of Chlamydiamicrovirus were studied with a view to identifying syntenic loci, i.e., similar genes in the same position in a genome of a group of genetically related phages, which suggested similar function. Small highly cationic peptides were identified that had a very similar molecular charge profile to previously identified antimicrobial peptides (AMPs). While the Chlamydia phage sequences had no protein sequence similarity to AMPs, lysins, or to known amurin proteins (such as Protein A2, protein E and others), the overall positive charge was a prominent feature. Using bioinformatic techniques as described above (JPRED and iTASSAR), structural predictions were conducted that revealed the presence of alpha helices, a hallmark feature of many AMPs. The alpha helices, the overall charge, the conservation among Chlamydia, and the related Gram-negative bacteria phage genomes all suggested that these proteins may represent a family of previously uncharacterized phage lytic polypeptides and that they may define a previously undescribed phage lytic mechanism. The fact that they were predicted to be small in size and soluble (based on their charge profile) also meant that, once synthesized, they would likely be readily amenable to testing by simply adding them to susceptible bacteria cultures.

Based on the foregoing, 12 conserved sequences within syntenic loci were extracted from the Microviridae genomes in the GenBank database and specifically from the Chlamydiamicroviruses genomes (as well as some other viruses described below). The 12 conserved sequences were annotated only as hypothetical, uncharacterized or non-structural proteins and encoded small (putatively) cationic proteins predicted to adopt alpha-helical structures. These 12 sequences are set forth in Table 1. One of the peptides in Table 1, Chp5, was synthesized to have a molecular charge different from Chp4 by replacing arginines and lysines, which are positively charged, with negatively charges amino acid residues. Chp5 was predicted to be inactive. While these peptides exhibit no sequence similarity to other lytic or antimicrobial proteins, they are predicted to adopt alpha-helical structures (for examples, see FIG. 1) similar to subsets of the large family of antibacterial agents AMPs. It was postulated that Chp peptides perform the host lysis function for the phages from which they are derived.

Based on the foregoing considerations, further study of genomes of other phages (related to the Chlamydiamicroviruses, in the same family, Microviridae) that infect Gram-negative bacteria, as well as other uncharacterized sources that presented with the same synteny and charge profile, yielded 14 additional peptides listed in Table 2. Together, all 39 peptides (excluding Chp5) form a related family of novel phage lytic agents. They are all from Microviridae sources.

Thus, a complete list of all Chp family members (including certain features of each peptide) is provided in Table 1 and Table 2. Included in this group are peptides Chp1-4 and 6-12 and CPAR39, which are derived from 11 different Chlamydiamicroviruses and are described in Table 1; peptides Chp2 and Chp3 are two identical peptides from two different phages. As stated above, Chp5 is a modified derivative of Chp4 generated by the replacement of all positively charged amino acids, including arginines and lysines, with negatively charged amino acids, including glutamine and glutamic acid. The additional 27 members of the Chp family were identified by homology with the Chlamydiamicrovirus proteins and are described in Table 2 (“Additional Chp family members”). The 27 additional Chp family members are not from Chlamydiamicrovirus sources but from putative Microviridae phage sources.

TABLE 1
Chlamydia phage (Chp)-derived lytic agents

| Protein | Identifier | Protein | pI/kDa | |
|---|---|---|---|---|
| name | Information | Sequence | (amino acids) | DNA Sequence |
| Chp1 | Phage Chp1 | MVRRRRLRR | 13.23/4669.64 | ATGGTTCGTAGAAGAC |
| | Gene: Chp1p08 | RISRRIFRRTV | (36) | GTTTGAGAAGAAGAA |
| | GenBank: | ARVGRRRRS | | TAAGTAGAAGAATTTT |
| | NP_044319.1 | FRGGIRF | | TAGAAGAACAGTAGC |
| | Family: | (SEQ ID NO: 1) | | TAGAGTTGGTAGAAG |
| | Microviridae | | | GCGAAGGTCTTTTCGT |
| | | | | GGTGGTATTAGATTTT |
| | | | | AA (SEQ ID NO: 27) |
| Chp2 | Phage 2 | MRLKMARRR | 12.90/5708.98 | ATGAGGTTAAAAATG |
| | Gene: Ch-2p5 | YRLPRRRSRR | (44) | GCACGAAGAAGATAC |
| | GenBank: | LFSRTALRM | | AGACTTCCGCGACGTA |
| | NP_054652.1 | HPRNRLRRIM | | GAAGTCGAAGACTTTT |
| | Family: | RGURF (SEQ | | TTCAAGAACTGCATTG |
| | Microviridae | ID NO: 2) | | AGGATGCATCCAAGA |
| | | | | AATAGGCTTCGAAGA |
| | | | | ATTATGCGTGGCGGCA |
| | | | | TTAGGTTCTAG (SEQ |
| | | | | lD NO: 28) |
| CPAR39 | Phage CPAR39 | MCKKVCKKC | 10.26/3993.91 | TTGTGCAAAAAAGTGT |
| | Gene: | PKKGPKNAP | (35) | GCAAAAAATGCCCAA |
| | CPA000S | KIGAFYERKT | | AAAAAGGGCCAAAAA |
| | GenBank: | PRLKQST | | ATGCCCCCAAAATCGG |
| | NP_063898.1 | (SEQ ID NO: 3) | | AGCATTTTACGAGAGA |
| | Family: | | | AAAACACCTAGACTTA |
| | Microviridae | | | AACAGTCTACTTGA |
| | | | | (SEQ ID NO: 29) |
| Chp3 | Phage 3 | MRLKMARRR | 12.90/5708.98 | ATGAGGTTAAAAATG |
| | Gene: CP3p6 | YRLPRRRSRR | (44) | GCACGAAGAAGATAC |
| | GenBank: | LFSRTALRM | | AGACTTCCGCGACGTA |
| | YP_022484.1 | HPRNRLRRIM | | GAAGTCGAAGACTTTT |
| | Family: | RGGIRF (same | | TTCAAGAACTGCATTA |
| | Microviridae | sequence as | | AGGATGCATCCAAGA |
| | | Chp2) (SEQ ID | | AATAGGCTTCGAAGA |
| | | NO: 54) | | ATTATGCGTGGCGGCA |
| | | | | TTAGGTTCTAG (SEQ |
| | | | | ID NO: 53) |
| Chp4 | Phage 4 | MARRYRLSR | 12.88/5073.11 | ATGGCACGAAGATAC |
| | Gene: Chp4p6 | RRSRRLFSRT | (39) | AGACTTTCGCGACGCA |
| | GenBank: | ALRMHRRNR | | GAAGTCGACGACTTTT |
| | YP_338243.1 | LRRIMRGGIR | | TTCAAGAACTGCATTA |
| | Family: | F (SEQ ID NO: | | AGAATGCATCGAAGA |
| | Microviridae | 4) | | AATAGACTTCGAAGA |
| | | | | ATTATGCGTGGCGGCA |
| | | | | TTAGGTTTTAG (SEQ ID |
| | | | | NO: 30) |
| Chp5 | Phage ChpQE | MAEQYELSQ | 3.73/4605.01 | ATGGCGGAACAGTAT |
| | Derivative of | EQSEQLFSET | (39) | GAACTGAGCCAGGAA |
| | Phage 4 | ALQMHEQNE | | CAGAGCGAACAGCTG |
| | | LQEIMQGGIE | | TTTAGCGAAACCGCGC |
| | | F (SEQ ID NO: | | TGCAGATGCATGAACA |
| | | 5) | | GAACGAACTGCAGGA |
| | | | | AATTATGCAGGGCGGC |
| | | | | ATTGAATTTTAA (SEQ |
| | | | | ID NO: 31) |
| Chp6 | Guinea pig | MARRRYRLP | 12.88/5180.27 | ATGGCACGAAGAAGA |
| | Chlamydia | RRRSRRLFSR | (40) | TACAGACTTCCGCGAC |
| | phage | TALRMHPRN | | GTAGAAGTCGAAGAC |
| | GenBank: | RLRRIMRGGI | | TTTTTTCAAGAACTGC |
| | NP_510878.1 | RF (SEQ ID | | ATTAAGGATGCATCCA |
| | Family: | NO: 6) | | AGAAATAGGCTTCGA |
| | Microviridae | | | AGAATTATGCGTGGCG |
| | | | | GCATTAGGTTCTAG |
| | | | | (SEQ ID NO: 32) |
| Chp7 | Uncharacterized | MKRRKMTRK | 12.31/4302.19 | ATGAAACGTAGAAAA |
| | protein | GSKRLFTATA | (38) | ATGACAAGAAAAGGT |
| | [Chlamydia | DKTKSINTAP | | TCTAAGCGTCTTTTTA |
| | trachomatis] | PPMRGGIRL | | CTGCAACTGCTGATAA |
| | GenBank: | (SEQ ID NO: 7) | | AACTAAATCTATCAAT |
| | CRH73061.1 | | | ACTGCCCCGCCGCCAA |
| | Family: | | | TGCGTGGCGGTATCCG |
| | Microviridae | | | GTTGTAA (SEQ ID NO: |
| | | | | 33) |
| Chp8 | Uncharacterized | MSKKRSRMS | 12.91/4672.61 | ATGTCTAAAAAGCGTT |
| | protein (C. | RRRSKKLFSK | (39) | CTCGCATGTCTCGCCG |
| | trachomatis) | TALRTKSVNT | | CCGTTCTAAGAAGTTG |
| | GenBank: | RPPMRGGFRF | | TTCTCGAAAACGGCTC |
| | CRH64983.1 | (SEQ ID NO: 8) | | TCCGCACGAAGAGTGT |
| | Family: | | | CAACACCCGTCCGCCT |
| | Microviridae | | | ATGCGCGGAGGGTTCC |
| | | | | GGTTCTGA (SEQ ID |
| | | | | NO: 34) |
| Chp9 | Uncharacterized | MSLRRHKLS | 12.91/4672.60 | ATGTCTCTTCGTCGTC |
| | protein (C. | RKASKRIFRK | (40) | ATAAGCTTTCTCGTAA |
| | trachomatis) | GASRTKTLNT | | GGCGTCTAAGCGTATT |
| | GenBank: | RATPMRGGF | | TTTCGTAAAGGTGCAT |
| | CRH84960.1 | RI (SEQ ID | | CACGCACGAAGACTTT |
| | Family: | NO: 9) | | GAATACTCGTGCTACG |
| | Microviridae | | | CCTATGCGCGGCGGTT |
| | | | | TCCGTATTTAA (SEQ ID |
| | | | | NO: 35) |
| Chp10 | Uncharacterized | MKRRKLSKK | 12.91/4570.64 | GTGAAACGTCGTAAAC |
| | protein (C. | KSRKIFTRGA | (38) | TGTCCAAAAAGAAATC |
| | trachomatis) | VNVKKRNLR | | TCGCAAGATTTTCACT |
| | GenBank: | ARPMRGGFRI | | CGCGGTGCTGTAAATG |
| | CRH93270.1 | (SEQ ID NO: | | TGAAAAAGCGTAACCT |
| | Family: | 10) | | TCGCGCTCGCCCAATG |
| | Microviridae | | | CGCGGCGGTTTCCGGA |
| | | | | TCTAA |
| | | | | (SEQ ID NO: 36) |
| Chp11 | Uncharacterized | MAKKMTKG | 11.74/4375.32 | ATGGCTAAAAAAATG |
| | protein (C. | KDRQVFRKT | (37) | ACTAAAGGCAAGGAT |
| | trachomatis) | ADRTKKLNV | | CGTCAGGTTTTTCGTA |
| | GenBank: | RPLLYRGGIR | | AAACCGCTGATCGTAC |
| | CRH59954.1 | L (SEQ ID NO: | | TAAGAAACTCAATGTT |
| | Family: | 11) | | AGACCGTTGTTATATC |
| | Microviridae | | | GAGGAGGTATCAGATT |
| | | | | ATGA (SEQ ID NO: 37) |
| Chp12 | Uncharacterized | MAGKKMVS | 11.74/4549.53 | ATGGCAGGAAAAAAA |
| | protein (C. | KGKDRQIFRK | (39) | ATGGTATCAAAAGGA |
| | trachomatis) | TADRTKKMN | | AAAGATAGACAGATTT |
| | GenBank: | VRPLLYRGGI | | TCCGAAAAACTGCTGA |
| | CRH59965.1 | RL (SEQ ID | | TCGCACTAAAAAAATG |
| | Family: | NO: 12) | | AATGTGCGCCCGCTAT |
| | Microviridae | | | TATATCGTGGAGGTAT |
| | | | | TAGATTATGA (SEQ ID |
| | | | | NO: 38) |

TABLE 2
Additional Chp family members

| Protein | Identifier | Protein | pI/kDa | |
|---|---|---|---|---|
| name | Information | Sequence | (amino acids) | DNA Sequence |
| Gkh1 | Marine | MRRPRKMNY | 12.66/4974.97 | ATGAGAAGACCAAGA |
| | gokushovirus | KKSKRMFSR | (41) | AAAATGAACTATAAA |
| | Gene: | TAARTHRKN | | AAATCAAAAAGAATG |
| | V508_gp1 | SLRGSRPMR | | TTTTCACGCACAGCAG |
| | GenBank: | GGIRL (SEQ | | CGAGAACACACAGAA |
| | YP_008798245.1 | ID NO: 13) | | AAAACTCTCTAAGAGG |
| | Unclassified | | | TAGCCGACCTATGAGA |
| | Gokushovirinae | | | GGCGGAATACGTCTTT |
| | | | | AA (SEQ ID NO: 39) |
| Gkh2 | Gokushovirinae | MSKKASRKS | 12.49/3794.63 | ATGTCGAAGAAGGCG |
| | Fen672_31 | FTKGAVKVH | (34) | TCGAGGAAGAGTTTTA |
| | Gene: | KKNVPTRVP | | CTAAGGGTGCCGTTAA |
| | AFL78_gp4 | MRGGIRL | | GGTTCATAAGAAAAAT |
| | GenBank: | (SEQ ID NO: | | GTTCCTACTCGTGTTC |
| | YP_009160382.1 | 14) | | CTATGCGTGGCGGTAT |
| | Unclassified | | | TAGGCTTTAG (SEQ ID |
| | Gokushovirinae | | | NO: 40) |
| Unp1 | Unnamed | MKMRKRTD | 12.31/4104.04 | ATGAAAATGCGTAAG |
| | protein product | KRVFTRTAA | (35) | CGGACGGACAAGCGA |
| | (uncultured | KSKKVNIAPK | | GTGTTTACCCGCACCG |
| | bacterium) | IFRGGIRL | | CTGCTAAGTCCAAGAA |
| | GenBank: | (SEQ ID NO: | | AGTGAACATTGCCCCG |
| | CDL66944.1 | 15) | | AAAATTTTTAGAGGAG |
| | Circular | | | GTATCCGTCTGTGA |
| | plasmid, rat | | | (SEQ ID NO: 41) |
| | cecum | | | |
| Ecp1 | Nonstructural | MARSRRRMS | 12.70/4812.72 | ATGGCTCGTTCTCGCC |
| | protein | KRSSRRSFRK | (39) | GTCGTATGTCCAAGCG |
| | (Escherichia | YAKTHKRNF | | TTCTTCCCGTCGTTCGT |
| | coli) | KARSMRGGI | | TCCGTAAGTACGCAAA |
| | GenBank: | RL (SEQ ID | | GACGCATAAACGTAA |
| | WP_100756432.1 | NO: 16) | | CTTTAAAGCCCGCTCT |
| | sEPEC Feces | | | ATGCGTGGTGGAATTC |
| | strain | | | GTCTTTGA (SEQ ID NO: |
| | | | | 42) |
| Tma1 | Hypothetical | MESPNSRSQL | 7.80/5433.39 | ATGGAAAGCCCGAAC |
| | protein (T. | GITLYLLSTIF | (47) | AGCCGCAGCCAGCTG |
| | maritimus) | PDACFRYRRE | | GGCATTACCCTGTATC |
| | SAMN04488044_0855 | LPYPLVIWGV | | TGCTGAGCACCATTTT |
| | GenBank: | ATLCLQ (SEQ | | TCCGGATGCGTGCTTT |
| | SHG47122.1 | ID NO: 17) | | CGCTATCGCCGCGAAC |
| | | | | TGCCGTATCCGCTGGT |
| | | | | GATTTGGGGCGTGGCG |
| | | | | ACCCTGTGCCTGCAGT |
| | | | | AA (SEQ ID NO: 43) |
| Ecp2 | Hypothetical | MARSRRRMS | 12.66/4770.68 | ATGGCTCGTTCCCGTA |
| | protein | KRSSRRSFRK | (39) | GACGTATGTCTAAGCG |
| | EC13107_44c00010 | YAKSHKKNF | | TTCTTCCCGCCGTTCG |
| | (E. coli) | KARSMRGGI | | TTCCGCAAGTATGCGA |
| | GenBank: | RL (SEQ ID | | AGTCGCATAAGAAGA |
| | OAC14041.1 | NO: 18) | | ACTTTAAAGCCCGCTC |
| | Udder, acute | | | AATGCGTGGCGGTATC |
| | mastitis | | | CGTTTATAA (SEQ ID |
| | | | | NO: 44) |
| Osp1 | Hypothetical | MRKRMSKRV | 11.90/4389.35 | ATGAGAAAGCGAATG |
| | protein | DKKVFRRTA | (37) | TCTAAGCGTGTTGACA |
| | SAMN05216343_103150 | ASAKKINIDP | | AGAAGGTGTTCCGTCG |
| | (Oscillibacter | KIYRGGIRL | | TACTGCCGCATCTGCC |
| | sp. PC13) | (SEQ ID NO: | | AAGAAGATTAACATTG |
| | GenBank: | 19) | | ACCCCAAGATTTACCG |
| | SFP13761.1 | | | TGGAGGTATTCGCCTA |
| | | | | TGA (SEQ ID NO: 45) |
| Unp2 | Unnamed | MRRRRLSRR | 13.18/4757.77 | ATGAGACGTCGTCGTC |
| | protein product | TSRRFFRKGL | (37) | TATCCCGCAGAACTTC |
| | GenBank: | KVRRRNLRA | | CCGCCGTTTTTTCCGT |
| | CDL65918.1 | RPMRGGFRI | | AAAGGACTTAAGGTTC |
| | Extrachromosomal | (SEQ ID NO: | | GCCGTCGTAACCTCCG |
| | DNA | 20) | | CGCGAGACCCATGAG |
| | RGI00327 | | | AGGCGGATTCAGAATT |
| | | | | TGA (SEQ ID NO: 46) |
| Unp3 | Unnamed | MARRKKMK | 12.32/4545.51 | ATGGCACGACGCAAG |
| | protein product | GKRDKRVFK | (39) | AAGATGAAAGGCAAG |
| | GenBank: | QTANKTKAI | | CGGGATAAACGGGTG |
| | CDL65808.1 | NISPKNMRG | | TTTAAGCAGACAGCCA |
| | Extrachromosomal | GTRL (SEQ ID | | ACAAAACCAAGGCTA |
| | DNA | NO: 21) | | TCAACATCAGCCCAAA |
| | RGI00234 | | | AAACATGAGAGGGGG |
| | | | | TACGAGACTGTGA |
| | | | | (SEQ ID NO: 47) |
| Gkh3 | Hypothetical | MLTVWSDTP | 11.2/6440.82 | ATGTTAACTGTGTGGA |
| | protein (Marine | TIKRRKDMY | (53) | GTGACACCCCTACCAT |
| | gokushovirus) | RKRMSRKKS | | AAAAAGGAGAAAAGA |
| | GenBank: | KKVFAKTAM | | CATGTATAGAAAGAG |
| | AGT39941.1 | KVNKRNHVK | | AATGTCAAGAAAGAA |
| | | PMRGGYRI | | AAGTAAAAAGGTTTTT |
| | | (SEQ ID NO: | | GCAAAAACCGCAATG |
| | | 22) | | AAAGTAAATAAAAGA |
| | | | | AACCACGTTAAACCTA |
| | | | | TGCGTGGTGGATATAG |
| | | | | AATATAA (SEQ ID NO: |
| | | | | 48) |
| Unp5 | Hypothetical | MMKYRKKM | 12.04/4536.61 | ATGATGAAGTACAGA |
| | protein (Marine | SAKSSRKQFT | (39) | AAAAAAATGAGCGCT |
| | gokushovirus) | KGAMKVKG | | AAAAGTAGCCGAAAG |
| | GenBank: | KNFTKPMRG | | CAATTTACAAAAGGCG |
| | AGT39924.1 | GIRL (SEQ ID | | CCATGAAAGTGAAGG |
| | | NO: 23) | | GTAAAAACTTCACAAA |
| | | | | ACCAATGCGCGGAGG |
| | | | | CATCCGTCTATAG |
| | | | | (SEQ ID NO: 49) |
| Unp6 | Hypothetical | MRRYNVNKG | 12.31/4492.34 | ATGCGACGTTACAATG |
| | protein (Marine | KSAKKFRKQ | (38) | TAAATAAAGGTAAATC |
| | gokushovirus) | VSKTKVANL | | TGCTAAGAAGTTTCGA |
| | GenBank: | RSNPMRGGW | | AAGCAGGTAAGTAAG |
| | AGT39915.1 | RL (SEQ ID | | ACGAAGGTTGCAAAC |
| | | NO: 24) | | CTACGTTCTAATCCAA |
| | | | | TGCGAGGTGGTTGGAG |
| | | | | ACTCTAA (SEQ ID NO: |
| | | | | 50) |
| Spi1 | Hypothetical | MAYRGFKTS | 12.37/3776.45 | ATGGCTTATCGTGGTT |
| | protein Sp-4p3 | RVVKHRVRR | (28) | TTAAAACGAGTCGTGT |
| | (Spiroplasma | RWFNHRRRY | | TGTAAAACATAGAGTA |
| | virus SpV4] | R (SEQ ID NO: | | CGTAGAAGATGGTTTA |
| | Orf9 | 25) | | ATCATAGAAGACGTTA |
| | NCBI Ref. Seq: | | | TAGATAG (SEQ ID NO: |
| | NP_598337.1 | | | 51) |
| Spi2 | Hypothetical | MRRKVKNTK | 12.91/4629.45 | GTGAGACGCAAGGTT |
| | protein Sp-4p2 | RHQWRLTHS | (38) | AAGAACACAAAGCGT |
| | (Spiroplasma | ARSIKRANIM | | CATCAGTGGAGGTTGA |
| | virus SpV4) | PSNPRGGRRF | | CTCATTCTGCACGTTC |
| | Orf8 | (SEQ ID: 26) | | AATTAAACGTGCTAAT |
| | NCBI Ref. Seq: | | | ATAATGCCGTCAAATC |
| | NP_598336.1 | | | CTCGTGGTGGACGTCG |
| | | | | TTTTTAG (SEQ ID NO: |
| | | | | 52) |
| Ecp3 | Nonstructural | MARSRRRMS | 12.76/4784.69 | ATGGCTCGTTCTCGTC |
| | protein | KRSSRRSFRK | (39) | GTCGTATGTCTAAACG |
| | (Escherichia) | YAKTHKKNF | | TTCTTCTCGTCGTTCTT |
| | NCBI Ref. Seq: | KARSMRGGI | | TTCGTAAATATGCTAA |
| | WP_105269219.1 | RL (SEQ ID | | AACTCATAAAAAAAAT |
| | | NO: 55) | | TTTAAAGCTCGTTCTA |
| | | | | TGCGTGGAGGAATTCG |
| | | | | TTTATAA (SEQ ID NO: |
| | | | | 68) |
| Ecp4 | Nonstructural | MARSRRRMS | 12.66/4770.68 | ATGGCGCGCAGCCGCC |
| | protein | KRSSRRSFRK | (39) | GCCGCATGAGCAAAC |
| | (Escherichia) | YAKSHKKNF | | GCAGCAGCCGCCGCA |
| | NCBI Ref. Seq: | KARSMRGGI | | GCTTTCGCAAATAT |
| | WP_105466506.1 | RL (SEQ ID | | GCGAAAAGCCATAAA |
| | | NO: 56) | | AAAAACTTTAAAGCGC |
| | | | | GCAGCATGCGCGGCG |
| | | | | GCATTCGCCTG (SEQ |
| | | | | ID NO: 69) |
| Lvp1 | Lysis protein | MSSTLCRWA | 9.7/6346.6 | TTGTCGTCAACCTTGT |
| | (Pseudomonas | VKALRCTRV | (55) | GCCGCTGGGCCGTTAA |
| | phage PP7) | YKEFIWKPLV | | GGCCCTGCGGTGTACC |
| | NCBI Ref. Seq: | ALSYVTLYLL | | CGTGTGTATAAGGAGT |
| | NP_042306.1 | SSVFLSQLSY | | TTATATGGAAACCCTT |
| | | PIGSWAV | | AGTAGCGCTCAGTTAC |
| | | (SEQ ID NO: | | GTGACGTTGTATCTTC |
| | | 57) | | TGAGCTCGGTCTTCCT |
| | | | | GTCCCAACTCAGCTAC |
| | | | | CCCATCGGGAGCTGGG |
| | | | | CGGTGTAG (SEQ ID |
| | | | | NO: 70) |
| (ABP1) | Lysis protein | MKKRTKALL | 9.93/4247.21 | ATGAAGAAAAGGACA |
| Lvp2 | (Acinetobacter | PYAVFIILSFQ | (35) | AAAGCCTTGCTTCCCT |
| | phage AP205) | LTLLTALFMY | | ATGCGGTTTTCATCAT |
| | NCBI Ref. Seq: | YHYTF (SEQ | | ACTCAGCTTTCAACTA |
| | NP_085469.1 | ID NO: 58) | | ACATTGTTGACTGCCT |
| | | | | TGTTTATGTATTACCA |
| | | | | TTATACCTTTTAG (SEQ |
| | | | | ID NO: 71) |
| ALCES1 | Hypothetical | MAKKIRNKA | 12.70/4599.52 | ATGGCAAAGAAAATT |
| | protein (Alces | RDRRIFTRTA | (38) | AGAAACAAAGCACGT |
| | alces faeces | SRMHKANRT | | GATAGACGTATCTTCA |
| | associated | PRFMRGGIRL | | CAAGAACAGCTTCACG |
| | microvirus | (SEQ ID NO: | | CATGCACAAGGCAAA |
| | MP12 5423) | 59) | | CCGCACACCAAGATTT |
| | NCBI Ref. Seq: | | | ATGAGAGGCGGTATTA |
| | AXB22573.1 | | | GGTTATGA (SEQ ID |
| | | | | NO: 72) |
| AVQ206 | Hypothetical | MRRKKMSRG | 13.10/4680.78 | ATGCGTCGTAAAAAA |
| | protein | KSKKLFRRTA | (38) | ATGTCACGCGGTAAAT |
| | (Gokushovirinae | KRVHRKNLR | | CAAAAAAACTCTTTCG |
| | environmental | ARPMRGGIR | | CCGAACAGCAAAACG |
| | samples) | M (SEQ ID | | CGTTCATCGAAAAAAC |
| | NCBI Ref. Seq: | NO: 60) | | CTACGAGCTCGCCCAA |
| | AVQ10236.1 | | | TGCGTGGCGGCATACG |
| | | | | CATGTAG (SEQ ID NO: |
| | | | | 73) |
| AVQ244 | Hypothetical | MAKRHKIPQ | 12.8/4566.43 | ATGGCGAAGCGACAC |
| | protein | RASQHSFTRH | (39) | AAAATCCCGCAACGC |
| | (Gokushovirinae | AQKVHPKNV | | GCGTCACAACATTCCT |
| | environmental | PRLPMRGGIR | | TCACGCGCCATGCGCA |
| | samples) | L (SEQ ID NO: | | AAAGGTCCACCCTAAG |
| | NCBI Ref. Seq: | 61) | | AACGTTCCCCGCCTGC |
| | AVQ10244.1 | | | CAATGCGAGGCGGTAT |
| | | | | CCGTCTCTAA (SEQ ID |
| | | | | NO: 74) |
| CDL907 | Unnamed | MRKKMHKSL | 11.96/4398.22 | ATGCGTAAAAAAATG |
| | protein product | DKRVFNRTA | (37) | CACAAATCATTAGACA |
| | (uncultured | KKSKKINVNP | | AGCGAGTGTTTAACCG |
| | bacterium) | VVYRGGIRL | | CACTGCAAAAAAATC |
| | NCBI Ref. Seq: | (SEQ ID NO: | | AAAAAAAATAAATGT |
| | CDL65907.1 | 62) | | TAATCCTGTAGTTTAT |
| | | | | CGTGGAGGTATTAGAT |
| | | | | TATGA (SEQ ID NO: 75) |
| AGT915 | Hypothetical | MRRYNVNKG | 12.41/4492.32 | ATGCGACGTTACAATG |
| | protein (Marine | KSAKKFRKQ | (38) | TAAATAAAGGTAAATC |
| | gokushovirus) | VSKTKVANL | | TGCTAAGAAGTTTCGA |
| | NCBI Ref. Seq: | RSNPMRGGW | | AAGCAGGTAAGTAAG |
| | AGT39915.1 | RL (SEQ ID | | ACGAAGGTTGCAAAC |
| | | NO: 63) | | CTACGTTCTAATCCAA |
| | | | | TGCGAGGTGGTTGGAG |
| | | | | ACTCTAA (SEQ ID NO: |
| | | | | 76) |
| HH3930 | Hypothetical | MRPVKRSRV | 12.95/4755.69 | ATGCGTCCAGTTAAAA |
| | protein | NKARSAGKF | (41) | GATCAAGAGTAAATA |
| | RINTHH_3930 | RKQVGKTKM | | AGGCCCGATCTGCAGG |
| | (Richelia | ANLRSNPMR | | CAAGTTTCGTAAGCAG |
| | intracellularis | GGWRL (SEQ | | GTCGGTAAAACAAAG |
| | HH01) | ID NO: 64) | | ATGGCAAATCTGCGTA |
| | NCBI Ref. Seq: | | | GTAATCCGATGCGCGG |
| | CCH66548.1 | | | CGGATGGCGGCTGTGA |
| | | | | (SEQ ID NO: 77) |
| Fen7875 | Hypothetical | MKPLKRKPV | 12.81/4699.7 | ATGAAGCCATTGAAGC |
| | protein | QKARSAAKF | (41) | GTAAGCCGGTTCAGAA |
| | (Gokushovirinae | RRNVSTVKA | | GGCGCGGTCAGCAGCC |
| | Fen7875_21) | ANMAVKPM | | AAGTTCCGTCGAAATG |
| | NCBI Ref. Seq: | RGGWRF | | TGTCTACCGTTAAGGC |
| | YP_009160399.1 | (SEQ ID NO: | | TGCCAATATGGCGGTG |
| | | 65) | | AAGCCGATGCGCGGC |
| | | | | GGTTGGCGGTTCTGA |
| | | | | (SEQ ID NO: 78) |
| SBR77 | Hypothetical | MTKRDIEYR | 11.48/4882.78 | ATGACCAAGAGAGAC |
| | protein | KALGLNPSEP | (44) | ATCGAGTACCGGAAA |
| | SEA_BABYRAY_77 | LPKIVGAVTR | | GCTTTGGGGCTCAACC |
| | (Mycobacterium | HGATLKRPR | | CATCTGAGCCGCTCCC |
| | phage | VTALAR (SEQ | | GAAGATTGTGGGTGCC |
| | BabyRay) | ID NO: 66) | | GTCACCCGCCACGGGG |
| | NCBI Ref. Seq: | | | CCACTCTGAAACGCCC |
| | AOT25441 | | | ACGGGTCACCGCACTG |
| | | | | GCCCGATAG (SEQ ID |
| | | | | NO: 79) |
| Bdp1 | Putative DNA | MKRKPMSRK | 12.9/5708.98 | ATGAAAAGAAAACCA |
| | binding protein | ASQKTFKKN | (38) | ATGAGCCGCAAGGCCT |
| | (Bdellovibrio | TGVQRMNHL | | CTCAAAAAACCTTCAA |
| | phage | NPRAMRGGI | | AAAGAACACAGGCGT |
| | phiMH2K) | RL (SEQ ID | | TCAACGCATGAACCAT |
| | NCBI Ref. Seq: | NO: 67) | | CTCAACCCACGCGCCA |
| | NP_073546.1 | | | TGCGTGGTGGCATTAG |
| | | | | ACTATAA (SEQ ID NO: |
| | | | | 80) |

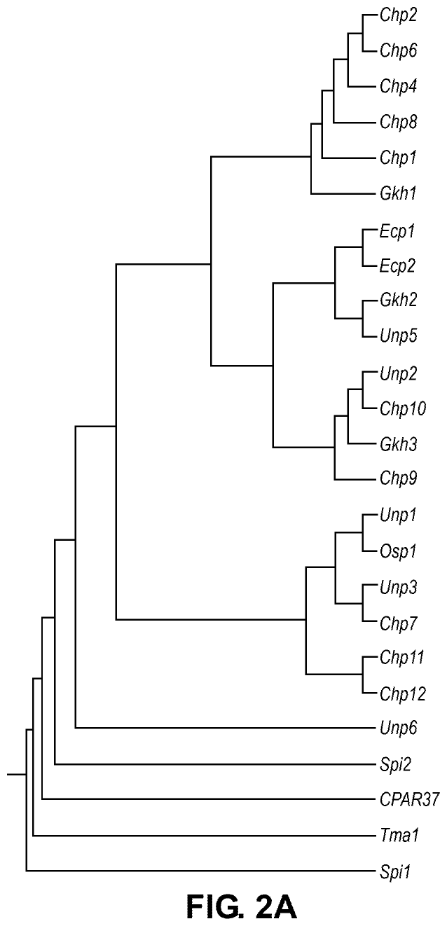

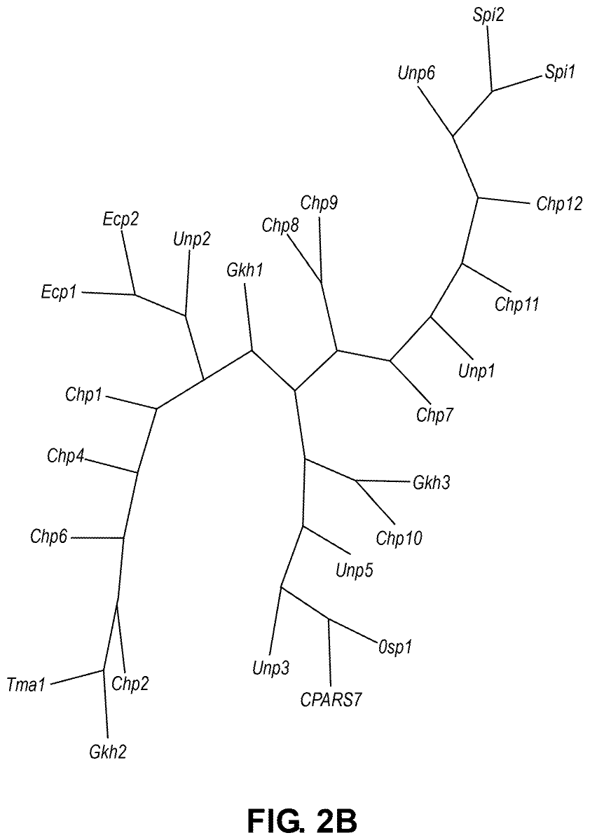

Additional information regarding the protein sequence homologies of several Chp family members is provided in Table 3. Chp1, Bdp1, Lvp1, and Lvp2 are the only Chp family members for which a predicted activity is indicated in the GenBank annotation. Chp1 (GenBank sequence NP_044319.1) is annotated as a DNA binding protein, although no data are provided to support this, and the annotation is inconsistent with a putative role in host lysis. Overall, the Chp proteins are 39-100% identical to each other and are not homologous to other peptides in the protein sequence database. Rooted and unrooted phylogenetic trees showing certain members of the Chp family are indicated in FIGS. 2A and 2B, respectively.

TABLE 3
Annotations and similarities of Chp family proteins

| Protein | Annotation (function) | Noted similarities |
|---|---|---|
| Chp1 | DNA binding protein Orf8; | 61.5% identical to Chp4 |
| | Mediates ssDNA packaging | 60% identical to Chp2 |
| | into virion; locates to the | 60% identical to Chp3 |
| | internal surface of the | Shared identity to others as well |
| | capsid; Plays role in viral | |
| | attachment to the host cell | |
| | (by similarity) | |
| Chp2 | Nonstructural protein | 60% identical to Chp1 |
| | | 100% identical to Chp3 |
| | | 92.5% identical to Chp4 |
| | | 55% identical to Chp8 |
| | | 54.8% identical to Gkh1 |
| | | 60.5% identical to Unp2 |
| | | Shared identity to others as well |
| CPAR39 | Uncharacterized protein | 60% identical to Chp6 |
| Chp3 | Nonstructural protein | 60% identical to Chp1 |
| | | 100% identical to Chp2 |
| | | 92.5% identical to Chp4 |
| | | 55% identical to Chp8 |
| | | 54.8% identical to Gkh1 |
| | | 60.5% identical to Unp2 |
| | | Shared identity to others as well |
| Chp4 | Putative structural protein | 61.5% identical to Chp1 |
| | | 92.5% identical to Chp2 |
| | | 92.5% identical to Chp3 |
| | | 55% identical to Gkh1 |
| | | 64.1% identical to Unp2 |
| | | 59.5% identical to Chp8 |
| | | Shared identity to others as well |
| Chp5 | Charge reversed variant of | RK residues from Chp4 changed |
| | Phage Chp4 Generated as a | to QE residues |
| | negative control protein | |
| Chp6 | Nonstructural protein | 60% identical to Chp1 |
| | | 100% identical to Chp2 (4 |
| | | residue truncation) |
| | | 100% identical to Chp3 (4 |
| | | residue truncation) |
| | | 92.5% identical to Chp4 |
| | | 55% identical to Chp8 |
| | | 54.8% identical to Gkh1 |
| | | 60.5% identical to Unp2 |
| | | Shared identity to others as well |
| Chp7 | Uncharacterized protein | 61.1% identical to Chp8 |
| | | 56. % identical to Unp3 |
| | | 50% identical to Chp9 |
| | | 53.7% identical to Gkh1 |
| | | 57.9% identical to Unp4 |
| | | Shared identity to others as well |
| Chp8 | Uncharacterized protein | 59% identical to Chp9 |
| | | 55% identical to Chp2 |
| | | 55% identical to Chp3 |
| | | 61.1% identical to Chp7 |
| | | 56.8% identical to Gkh3 |
| | | 59.5% identical to Chp4 |
| | | 47.2% identical to Chp10 |
| | | 50% identical to Gkh2 |
| | | 47.4% identical to Unp5 |
| | | 46.2% identical to Gkh1 |
| | | Shared identity to others as well |
| Chp9 | Uncharacterized protein | 59% identical to Chp8 |
| | | 59% identical to Unp2 |
| | | 57.9% identical to Chp10 |
| | | 50% identical to Chp7 |
| | | 46.2% identical to Unp6 |
| | | Shared identity to others as well |
| Chp10 | Uncharacterized protein | 63. % identical to Unp2 |
| | | 52.6% identical to Gkh2 |
| | | 57.9% identical to Chp9 |
| | | 61.8% identical to Gkh2 |
| | | 56.4% identical to Unp5 |
| | | 51.3% identical to Chp4 |
| | | 47.5% identical to Chp2 |
| | | 47.5% identical to Chp3 |
| | | 47.4% identical to Chp7 |
| | | 47.2% identical to Chp8 |
| | | 44.4% identical to Chp1 |
| | | Shared identity to others as well |
| Chp11 | Uncharacterized protein | Similar to above |
| Chp12 | Uncharacterized protein | Similar to above |
| Gkh1 | Uncharacterized protein | 55% identical to Chp4 |
| | | 54.8% identical to Chp2 |
| | | 54.8% identical to Chp3 |
| | | 53.7% identical to Chp7 |
| | | 48.8% identical to Chp10 |
| | | 46.2% identical to Chp8 |
| | | 40.5% identical to Gkh3 |
| | | 42.5% identical to Chp1 |
| | | Shared identity to others as well |
| Gkh2 | Uncharacterized protein | 70.6% identical to Unp5 |
| | | 63.6% identical to Chp10 |
| Unp1 | Unnamed protein product | 70.6% identical to Osp1 |
| | | 57.9% identical to Chp7 |
| | | 42.4% identical to Chp1 |
| | | 39.5% identical to Chp10 |
| | | 45.2% identical to Chp4 |
| | | 41.2% identical to Gkh2 |
| | | 45.2% identical to Chp2 |
| | | Shared identity to others as well |
| Ecp1 | Nonstructural protein | 60% identical to Unp2 |
| | | 56.4% identical to Chp4 |
| | | 53.8% identical to Chp2 |
| | | 53.8% identical to Chp3 |
| | | 61.8% identical to Gkh2 |
| | | 50% identical to Chp10 |
| | | 50% identical to Unp5 |
| | | 51.3% identical to Chp1 |
| | | Shared identity to others as well |
| Ecp2 | Hypothetical protein | 57.1% identical to Unp2 |
| | | 64.7% identical to Gkh2 |
| | | 53.8% identical to Chp4 |
| | | 51.3% identical to Chp2 |
| | | 51.3% identical to Chp3 |
| | | 52.8% identical to Osp1 |
| | | 47.2% identical to Chp10 |
| | | 47.5% identical to Chp1 |
| | | Shared identity to others as well |
| Tma1 | Uncharacterized protein | None |
| Osp1 | Hypothetical protein | 70.6% identical to Unp1 |
| | | 37.1% identical to Chp1 |
| | | 48.6% identical to Chp8 |
| | | 48.6% identical to Gkh3 |
| | | Shared identity to others as well |
| Unp2 | Unnamed protein product | 63.2% identical to Chp10 |
| | | 59% identical to Chp9 |
| | | 64.1% identical to Chp4 |
| | | 56.8% identical to Chp1 |
| | | 60.5% identical to Chp2 |
| | | 60.5% identical to Chp3 |
| | | Shared identity to others as well |
| Unp3 | Unnamed protein product | 56.8% identical to Chp7 |
| | | 58.8% identical to Unp1 |
| | | 59.5% identical to Osp1 |
| | | 43.2% identical to Chp9 |
| | | 45.9% identical to Gkh3 |
| | | Shared identity to others as well |
| Gkh3 | Uncharacterized protein | 52.6% identical to Chp10 |
| | | 55.9% identical to Chp8 |
| | | 50% identical to Unp2 |
| | | 42.9% identical to Chp4 |
| | | 47.2% identical to Chp9 |
| | | 40% identical to Chp2 |
| | | 40% identical to Chp3 |
| | | Shared identity to others as well |
| Unp5 | Uncharacterized protein | 61.1% identical to Gkh3 |
| | | 56.4% identical to Chp10 |
| | | 70.6% identical to Gkh2 |
| | | 53.8% identical to Chp7 |
| | | 43.6% identical to Unp2 |
| | | 48.6% identical to Chp9 |
| | | Shared identity to others as well |
| Unp6 | Uncharacterized protein | 46.2% identical to Chp9 |
| | | 44.7% identical to Chp10 |
| | | Shared identity to others as well |
| Spi1 | Hypothetical protein | No homology |
| Spi2 | Hypothetical protein | No homology |

### Example 2

### Synthesis of the Chp Peptides

All Chp peptides were synthesized by GenScript, NJ, USA with capping [N-terminal acetylation (Ac) and C-terminal amidation (NH₂)] on a fee-for-service basis. GenScript assessed the purity of each peptide by high performance liquid chromatography (HPLC) and mass spectrometry (MS). GenScript also performed a solubility test for all peptides and determined the net peptide content (NPC %) using a Vario MICRO Organic Elemental Analyzer. With the exception of Chp5, Lvp1, and Lvp2, all peptides were soluble in water and were suspended at a concentration of either 5 mg/mL or 10 mg/mL. Chp5 and Lvp1 were suspended in DMSO at a concentration of 10 mg/mL; Lvp2 was suspended in DMSO at a concentration of 2 mg/mL. Control peptides RI18, RP-1, WLBU2, BAC3, GN-2 amp, GN-3 amp, GN-4 amp, GN-6 amp, and Bac8c were also synthesized at GenScript as above. All additional peptides were commercial products purchased from either GenScript or Anaspec.

### Example 3

### Activity of Chp Peptides—Minimum Inhibitory Concentration (MIC) Against Gram-Negative Bacteria

The 39 Chp peptides (excluding Chp3, which has an identical peptide sequence to Chp2) were synthesized and examined in antimicrobial susceptibility testing (AST) formats. First, MIC values were determined against the carbapenam-resistant P. aeruginosa clinical isolate CFS-1292 in CAA medium supplemented with 2.5% human serum (Table 4). Several peptides, including Chp1, Chp2, Chp4, Chp6, CPAR39 (with dithiothreitol (DTT)), Chp7, Chp8, Chp10, Chp11, Ecp1, Ecp2, Osp1, Spil, Gkh3, Unp2, Unp5, Unp6, Ecp3, Ecp4, Lvp1, ALCES1, AVQ206, CDL907, AGT915, and SBR77, exhibited superior MIC values ranging from 0.25-4 μg/mL. Peptides Chp5, CPAR39 (without DTT), Gkh1, Unp1, Spi2, and Bdp1 were only poorly active and exhibited MIC values of >32 μg/mL. CPAR39 is unique in this group as it contains internal cysteine residues and requires the presence of 0.5 mM DTT for activity. Chp5 was designed as a derivative of Chp4 in which all positively charged residues were changed to negative charges; it is predicted, based on studies of cationic AMPs, that cationic residues are required for the antibacterial activity and removal of the cationic residues with anionic residues will ablate activity. Accordingly, Chp5 (MIC>64 μg/mL) is an inactive variant of Chp4 (MIC=0.5 μg/mL). Both CPAR39 (without DTT) and Chp5 are used as negative controls.

TABLE 4

| | MIC (μg/mL) |
|---|---|
| Peptide | against CFS-1292 |
| Chp1 | 2 |
| Chp2 | 0.5 |
| CPAR39 + DTT | 4 |
| CPAR39 − DTT | 64 |
| Chp4 | 0.5 |
| Chp5 | >64 |
| Chp6 | 0.25 |
| Chp7 | 4 |
| Chp8 | 2 |
| Chp9 | 8 |
| Chp10 | 2 |
| Chp11 | 4 |
| Chp12 | 8 |
| Gkh1 | 128 |
| Gkh2 | 8 |
| Gkh3 | 2 |
| Unp1 | 32 |
| Unp2 | 1 |
| Unp3 | 8 |
| Unp5 | 2 |
| Unp6 | 4 |
| Ecp1 | 0.5 |
| Tma1 | n.d. |
| Ecp2 | 1 |
| Osp1 | 0.5 |
| Spi1 | 2 |
| Spi2 | 64 |
| Ecp3 | 4 |
| Ecp4 | 2 |
| Bdp1 | >128 |
| Lvp1 + DTT | 2 |
| Lvp2 | 8 |
| ALCES1 | 2 |
| AVQ206 | 2 |
| AVG244 | >16 |
| CDL907 | 2 |
| AGT915 | 1 |
| HH3930 | n.d. |
| Fen7875 | n.d. |
| SBR77 | 0.5 |

Additional MIC testing was performed using peptides Chp1, Chp2, Chp4, CPAR39 (without DTT), Chp6, Ecp1 and Ecp2 against a range of Gram-negative organisms including Pseudomonas aeruginosa, Escherichia coli, Enterobacter cloacae, Klebsiella pneumoniae, and Acinetobacter baumannii, which includes certain major ESKAPE pathogens (Table 5). Testing was performed in CAA (containing physiological salt concentrations) that was not supplemented with 2.5% human serum, owing to the differential susceptibilities of target organisms to the presence of human serum. Superior MIC values of 1-4 μg/mL were observed against all strains tested for Chp2, Chp4, Chp6, Ecpl, and Ecp2, indicating broad spectrum activity for the present Chp peptides in the context of physiological salt concentrations. Chp2 and Ecp1 were additionally tested against Salmonella typhimurium and demonstrated to have an MIC of 2 μg/mL.

TABLE 5

| | | MIC (μg/mL) | | | | | | |
|---|---|---|---|---|---|---|---|---|
| | | | CPAR39 − | | | | | |
| RSC | Organism (strain number) | Chp2 | DTT | Chp4 | Ecp1 | Chp6 | Chp1 | Ecp2 |
| 489 | P. aeruginosa (ATCC | 4 | 128 | 4 | 2 | 2 | 4 | 2 |
| | 15692, infected wound) | | | | | | | |
| 490 | P. aeruginosa (PAO1, | 4 | 128 | 4 | 2 | 1 | 8 | 2 |
| | alternate source, | | | | | | | |
| | HER1018) | | | | | | | |
| 815 | P. aeruginosa (ATCC | 4 | >128 | 4 | 2 | 1 | 8 | 2 |
| | 27853, MIC control | | | | | | | |
| | strain) | | | | | | | |
| 1108 | P. aeruginosa (ATCC | 2 | 16 | 4 | 1 | 2 | 8 | 4 |
| | 19142, tracheobronchial | | | | | | | |
| | secretion) | | | | | | | |
| 1109 | P. aeruginosa (ATCC | 4 | >128 | 4 | 2 | 4 | 8 | 4 |
| | 17646, human liver | | | | | | | |
| | abscess) | | | | | | | |
| 1110 | P. aeruginosa (ATCC | 4 | 128 | 4 | 1 | 2 | 4 | 1 |
| | 15152, abscess in middle | | | | | | | |
| | ear) | | | | | | | |
| 1111 | P. aeruginosa (ATCC | 4 | >128 | 4 | 2 | 4 | 8 | 4 |
| | 14213, human hip | | | | | | | |
| | wound) | | | | | | | |
| 1113 | P. aeruginosa (ATCC | 4 | >128 | 4 | 4 | 2 | 8 | 2 |
| | BAA-27, lab strain) | | | | | | | |
| 1114 | P. aeruginosa (ATCC | 4 | >128 | 4 | 2 | 2 | 8 | 2 |
| | 25102, bacteriophage | | | | | | | |
| | host) | | | | | | | |
| 1115 | P. aeruginosa (ATCC | 4 | 128 | 4 | 2 | 4 | 8 | 8 |
| | 15692, infected wound) | | | | | | | |
| 1292 | P. aeruginosa 453 | 4 | 16 | 4 | 2 | 2 | 8 | 8 |
| | (Human clinical isolate, | | | | | | | |
| | HSS) | | | | | | | |
| 813 | E. coli (ATCC 25922, | 2 | 32 | 2 | 2 | 2 | 8 | 2 |
| | MIC control strain) | | | | | | | |
| 1212 | E. coli (HM346, colon, | 2 | 16 | 4 | 2 | 2 | 8 | 4 |
| | Crohn's disease) | | | | | | | |
| 1240 | Enterobacter cloacae | 4 | >128 | 4 | 2 | 4 | 8 | 4 |
| | (ATCC 13047, MIC | | | | | | | |
| | control strain) | | | | | | | |
| 814 | Klebsiella pneumoniae | 4 | 64 | 4 | 2 | 4 | 16 | 4 |
| | (ATCC 10031 (MIC | | | | | | | |
| | control strain) | | | | | | | |
| 1131 | Klebsiella spp. (HM-223; | 4 | >128 | 4 | 2 | 4 | 8 | 2 |
| | gut of Crohn's disease | | | | | | | |
| | patient) | | | | | | | |
| 1138 | Klebsiella pneumoniae | 4 | >128 | 4 | 2 | 4 | 16 | 2 |
| | (g2-3 HM35K) | | | | | | | |
| 1139 | Klebsiella sp. (HM-44; | 2 | 64 | 4 | 2 | 1 | 8 | 4 |
| | colon, Crohn's disease) | | | | | | | |
| 30 | Acinetobacter baumannii | 2 | 64 | 4 | 2 | 2 | 8 | 2 |
| | (clinical isolate HSS) | | | | | | | |
| 32 | Acinetobacter baumannii | 2 | 64 | 4 | 2 | 2 | 8 | 2 |
| | (clinical isolate HSS) | | | | | | | |
| 27 | Salmonella typhimurium | 2 | n.d. | n.d. | 2 | n.d. | n.d. | n.d. |
| | LT2 (lab isolate) | | | | | | | |

n.d. = not determined

The MIC values for both Chp2 and Chp4 were also determined and compared to that of a range of AMPs from the literature (including innate immune effectors and derivatives thereof), against the laboratory P. aeruginosa strain PAO1 in Mueller-Hinton broth supplemented with either 50% human plasma or human serum (Table 6). Here, the use of PAO1 (a laboratory isolate) enables testing in the presence of elevated serum or plasma concentrations; PAO1, unlike most clinical isolates, is insensitive to the antibacterial activity of human blood matrices. In Table 6, the MIC values for Chp2 and Chp4 were 2 μg/mL; in comparison, only RI18 and protegrin were similarly active (MIC =1-4 μg/mL), and the 18 additional peptides tested were either inactive or poorly active.

TABLE 6

Minimal inhibitory
concentration (μg/mL)

| | Human | Human |
|---|---|---|
| Agent | Plasma | Serum |
| Protegrin 1 | 1 | 4 |
| Indolicidin | >64 | >64 |
| LL-37 | >64 | >64 |
| LL-37 (18-37) | >64 | >64 |
| LL-37 (17-29) | >64 | >64 |
| GN-2 amp | >64 | >64 |
| GN-3 amp | >64 | >64 |
| GN-4 amp | >64 | >64 |
| Pediocin | >64 | >64 |
| Parasin | >64 | >64 |
| PGLa | >64 | >64 |
| OV-1 | 32 | 32 |
| Dermaseptin | >64 | >64 |
| WLBU2 | >64 | >64 |
| RP-1 | 32 | 64 |
| T9W | 16 | 32 |
| BAC3 | >64 | >64 |
| GN-6 amp | >64 | >64 |
| Bac 8c | >64 | >64 |
| RI18 | 2 | 1 |
| Chp2 | 2 | 2 |
| Chp4 | 2 | 2 |

### Example 4

### Activity of Chp Peptides—Eradication of Biofilm of Gram-Negative Bacteria

To evaluate anti-biofilm activity, MBEC (minimum biofilm eradication concentration) values were determined for peptides Chp2 and Chp4 against mature biofilms formed by P. aeruginosa strain ATCC 17647 in tryptic soy broth medium supplemented with 2% glucose. MBEC values of 0.25 μg/mL were observed for both Chp2 and Chp4 (Table 7), which are consistent with a potent ability to eradicate mature biofilms. In comparison, the activity of RI18, a highly active AMP (15), was observed to be substantially lower, 4 μg/mL, and the activity of T4 lysozyme, a poorly active lysin, was observed to be >64 μg/mL.

TABLE 7

| Agent | MBEC (μg/mL) |
|---|---|
| RI18 | 4 |
| Chp2 | 0.25 |
| Chp4 | 0.25 |
| T4LYZ | >64 |

### Example 5

### Combination of Chp Peptides and Antibiotics

To evaluate synergy between either Chp2 or Chp4 and a range of 11 antibiotics, each combination of Chp2 with the 11 antibiotics and Chp4 with the 11 antibiotics was tested in a standard checkerboard assay format using P. aeruginosa strain CFS-1292 in CAA media supplemented with 2.5% human serum. In the checkerboard assay, fractional inhibitory concentration index (FICI) values are calculated. FICI values≤0.5 are consistent with synergy, values>0.5-1 are consistent with strongly additive activity, values of 1-2 are consistent with additive activity, and values>2 are considered antagonistic. As shown below in Table 8, for both Chp2 and Chp4, the values were consistent with either synergy (i.e., ≤0.5) or strongly additive (i.e., >0.5-1) interactions between the Chp peptide and the antibiotic.

TABLE 8

| Antibiotic | Chp2 | Chp4 |
|---|---|---|
| Amikacin | 0.500 | 0.500 |
| Azithromycin | 0.156 | 0.156 |
| Aztreonam | 0.500 | 0.375 |
| Ciprofloxacin | 0.500 | 0.375 |
| Colistin | 0.375 | 0.375 |
| Fosfomycin | 0.250 | 0.250 |
| Gentamicin | 0.281 | 0.250 |
| Imipenem | 0.188 | 0.375 |
| Piperacillin | 0.188 | 0.188 |
| Rifampicin | 0.563 | 0.750 |
| Tobramycin | 0.266 | 0.266 |

### Example 6

### Assessment of Hemolytic Activity of Chp Peptides

Antimicrobial peptides amenable for use in treating invasive infections should show low toxicity against erythrocytes (Oddo A. et al, 2017. Methods Mol Biol 1548:427-435). To examine the potential for hemolytic activity, a common methodology (described in Materials and Methods above) was used for measuring the ability of AMPs to lyse red blood cells based on the determination of minimal hemolytic concentrations (MHCs) against human red blood cells. For 33 of the 37 Chp peptides tested, no evidence of hemolysis was observed, with MHC values of >128 μg/mL (Table 9). Triton X100 control was tested at a starting concentration of 2%, and the MHC was observed at 0.007%. In comparison, four AMPs with known hemolytic activity, including RI18, R12, RR12p, and RR12h, were observed with MHC values ranging from 4-128 μg/mL. Triton X-100, a membranolytic detergent commonly used as a positive control in hemolytic assays, was hemolytic over a range of concentrations from 2% to 0.007%. These findings suggest that Chp peptides do not have the in vitro toxicity (i.e., hemolytic activity) commonly observed for AMPs. This property is expected of the remaining Chp peptides of Tables 1 and 2 based not only on percent sequence identity, 3D structural similarity, and charge profile, but also on the anticipation that, as lytic agents, the present peptides will most likely be very highly specific for the Gram-negative cell envelope.

TABLE 9
Minimal hemolytic concentration (MHC) values
determined against human red blood cells

| | |
|---|---|
| Agent | MHC (μg/mL) |

Control Peptides

| | |
|---|---|
| RI18 | 128 |
| RR12 | 8 |
| RR12p | 4 |
| RR12h | 32 |
| Triton control* | 1 |

Chp Peptides

| | |
|---|---|
| Chp1 | >128 |
| Chp2 | >128 |
| CPAR39 | >128 |
| Chp4 | >128 |
| Chp5 | >128 |
| Chp6 | >128 |
| Chp7 | >128 |
| Chp8 | >128 |
| Chp9 | >128 |
| Chp10 | >128 |
| Chp11 | >128 |
| Chp12 | >128 |
| Gkh1 | >128 |
| Gkh2 | >128 |
| Gkh3 | >128 |
| Ecp1 | >128 |
| Ecp2 | >128 |
| Ecp3 | >128 |
| Ecp4 | >128 |
| Osp1 | >128 |
| Unp1 | >128 |
| Unp2 | >128 |
| Unp3 | >128 |
| Unp5 | >128 |
| Unp6 | >128 |
| Spi1 | >128 |
| Spi2 | >128 |
| Bdp1 | >128 |
| Lvp1 | n.d. |
| Lvp2 | 8 |
| ALCES1 | >128 |
| AVQ206 | >128 |
| AVQ244 | >128 |
| CDL907 | >128 |
| AGT915 | >128 |
| HH3930 | n.d. |
| Fen7875 | n.d. |
| SBR77 | >128 |

### Example 7

### Duration of Lytic Activity Against Gram Negative Bacteria

The activity of Chp2 and Chp4 was examined against P. aeruginosa strain CFS-1292 in the time-kill format using CAA with 2.5% human serum as described in Materials and Methods. Assessments of bacterial viability at 1, 3, and 24 hours after treatment with 1 μg/mL and 10 μg/mL concentrations of either Chp2 or Chp4 resulted in multi-log fold decreases consistent with potent bactericidal activity in all cases (Table 10). Table 10 sets forth the log reduction of colony forming units (compared to untreated controls) determined using the time-kill format for P. aeruginosa strain CFS-1292 after treatment in CAA supplemented with 2.5% human serum.

TABLE 10

| | Loss of bacterial viability (log10 CFU/mL) | | |
|---|---|---|---|
| Treatment | 1 hour | 3 hours | 24 hours |
| Chp2 (1 μg/mL) | >3.5 | >4 | >4.8 |
| Chp2 (10 μg/mL) | >3.5 | >4 | >4.8 |
| Chp4 (1 μg/mL) | >3.5 | >4 | >4.8 |
| Chp4 (10 μg/mL) | >3.5 | >4 | >4.8 |

Additionally, a stability assessment was conducted to detect the fold change in MIC after incubation of peptides prepared as described above in Example 2. Stability was assessed after incubation in 100% human serum at 37° C. after 10 minutes, 1 hour, and 2 hours. The results are shown below in Table 11.

TABLE 11

| | Fold change in MIC | | |
|---|---|---|---|
| Peptide | 10 minutes | 1 hour | 2 hours |
| Chp1 | 1 | 1 | 1 |
| Chp2 | 1 | 1 | 2 |
| CPAR39 | 1 | 0.5 | 0.5 |
| Chp4 | 1 | 1 | 0.5 |
| Chp5 | 1 | 2 | 2 |
| Chp6 | 1 | 1 | 1 |
| Chp7 | 1 | 1 | 1 |
| Chp8 | 1 | 1 | 1 |
| Chp9 | 1 | 0.5 | 0.5 |
| Chp10 | 1 | 2 | 2 |
| Chp11 | 1 | 2 | 2 |
| Chp12 | 1 | 1 | 1 |
| Gkh1 | 1 | 0.5 | 1 |
| Gkh2 | 1 | 0.5 | 2 |
| Gkh3 | 1 | 1 | 1 |
| Ecp1 | 1 | 4 | 1 |
| Ecp2 | 1 | 2 | 2 |
| Ecp3 | 1 | 1 | 1 |
| Ecp4 | n.d. | n.d. | n.d. |
| Osp1 | 1 | 0.5 | 1 |
| Unp1 | 1 | 0.5 | 2 |
| Unp2 | 1 | 2 | 1 |
| Unp3 | 1 | 1 | 1 |
| Unp5 | 1 | 1 | 4 |
| Unp6 | 1 | 1 | 1 |
| Spi1 | 1 | 2 | 2 |
| Spi2 | 1 | 1 | 1 |
| Bdp1 | 1 | 1 | 0.25 |
| Lvp1 | n.d. | n.d. | n.d. |
| Lvp2 | 1 | 1 | 0.25 |
| ALCES1 | 1 | 1 | 1 |
| AVQ206 | 1 | 1 | 1 |
| AVQ244 | 1 | 1 | 0.5 |
| CDL907 | 1 | 1 | 1 |
| AGT915 | 1 | 1 | 0.5 |
| HH3930 | n.d. | n.d. | n.d. |
| Fen7875 | n.d | n.d. | n.d |
| SBR77 | 1 | 4 | 1 |

As shown in Table 11, all of Chp1, Chp2, CPAR39, Chp4, Chp5, Chp6, Chp7, Chp8, Chp9, Chp10, Chp11, Chp12, Gkh1, Gkh2, Gkh3, Ecp1, Ecp2, Ecp3, Ecp4, Osp1, Unpl, Unp2, Unp3, Unp5, Unp6, Spi1, Spi2, Bdp1, Lvp1, Lvp2, ALCES1, AVQ206, AVQ244, CDL907, AGT915, HH3930, Fen7875, and SBR77 were adequately stable after 10 minutes, 1 hour, and 2 hours.

1. A pharmaceutical composition comprising:
an effective amount of (i) an isolated Chp peptide having an amino acid sequence selected from the group consisting of SEQ ID NOs. 1-4, 6-26 and 54-66 or active fragment thereof, or (ii) a modified Chp peptide having 80% sequence identity with the amino acid sequence of at least one of SEQ ID NOs. 1-4, 6-26 and 54-66, wherein the modified Chp peptide inhibits the growth, reduces the population, or kills at least one species of Gram-negative bacteria; and
a pharmaceutically acceptable carrier
wherein the Chp peptide contains at least one non-natural modification relative to the amino acid sequence of any one of SEQ ID NOs. 1-4, 6-26 and 54-66 or active fragments thereof.

2. (canceled)

3. The pharmaceutical composition of claim 1, wherein the non-natural modification is selected from the group consisting of substitution modifications, N-terminal acetylation modifications, and C-terminal amidation modifications.

4. (canceled)

5. (canceled)

6. The pharmaceutical composition according to claim 1, which is a solution, a suspension, an emulsion, an inhalable powder, an aerosol, or a spray.

7. The pharmaceutical composition according to claim 1, further comprising one or more antibiotics suitable for the treatment of Gram-negative bacteria.

8. A vector comprising a nucleic acid molecule that encodes (i) a Chp peptide having an amino acid sequence selected from the group consisting of SEQ ID NOs. 1-4, 6-26 and 54-66 or active fragments thereof, or (ii) a modified Chp peptide having 80% sequence identity with the amino acid sequence of at least one of SEQ ID NOs. 1-4, 6-26 and 54-66, wherein the modified Chp peptide inhibits the growth, or reduces the population, or kills at least one species of Gram-negative bacteria.

9. The vector according to claim 8, wherein the vector is a recombinant expression vector and wherein the nucleic acid is operatively linked to a heterologous promoter.

10. (canceled)

11. (canceled)

12. The vector of claim 8, wherein the nucleic acid molecule is a cDNA sequence.

13. An isolated host cell comprising the vector of claim 8.

14. An isolated, purified nucleic acid encoding a Chp peptide comprising an amino acid sequence selected from the group consisting of SEQ ID NOs. 1-4, 6-26 and 54-66 or active fragments thereof or a nucleic acid comprising a sequence complementary thereto, wherein the Chp peptide contains at least one non-natural modification relative to the amino acid sequence of any one of SEQ ID NOs. 1-4, 6-26 and 54-66 or active fragments thereof.

15. (canceled)

16. The isolated, purified nucleic acid of claim 14 wherein the non-natural modification is selected from the group consisting of substitution modifications, N-terminal acetylation modifications, and C-terminal amidation modifications.

17. An isolated, purified DNA comprising a nucleotide sequence selected from the group consisting of SEQ ID NOs. 27-30, SEQ ID NOs. 32-53, and SEQ ID NOs. 68-79 wherein the nucleotide sequence contains at least one non-natural modification.

18. (canceled)

19. The isolated, purified DNA of claim 17, wherein the non-natural modification is a mutation or a nucleic acid sequence encoding an N-terminal acetylation modification or a C-terminal amidation modification.

20. A method of inhibiting the growth, reducing the population, or killing of at least one species of Gram-negative bacteria, the method comprising contacting the bacteria with a composition comprising an effective amount of (i) a Chp peptide comprising an amino acid sequence selected from the group consisting of SEQ ID NOs. 1-4, 6-26 and 54-66 or active fragments thereof, or (ii) a modified Chp peptide having 80% sequence identity with the amino acid sequence of at least one of SEQ ID NOs. 1-4, 6-26 and 54-66, said Chp peptide or modified Chp peptide having lytic activity for a period of time sufficient to inhibit said growth, reduce said population, or kill said at least one species of Gram-negative bacteria.

21. (canceled)

22. (canceled)

23. A method of preventing or treating a bacterial infection caused by at least one species of Gram-negative bacteria, comprising administering to a subject diagnosed with, at risk for, or exhibiting symptoms of a bacterial infection, a pharmaceutical composition according to claim 1.

24. The method of claim 20, wherein the Gram-negative bacteria is selected from the group consisting of Acinetobacter baumannii, Pseudomonas aeruginosa, Escherichia coli, Klebsiella pneumoniae, Enterobacter cloacae, Salmonella, Neisseria gonorrhoeae, and Shigella.

25. The method of claim 20, wherein the at least one species of Gram-negative bacteria is Pseudomonas aeruginosa.

26. The method of claim 20, wherein the bacterial infection is a topical or systemic bacterial infection.

27. The method of claim 23, further comprising administering to the subject an antibiotic suitable for the treatment of Gram-negative bacterial infection.

28. The method of claim 27, wherein the antibiotic is selected from one or more of azithromycin, aztreonam, fosfomycin, ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

29. (canceled)

30. The method of claim 27, wherein administering the pharmaceutical composition is more effective in inhibiting the growth, reducing the population, or killing the Gram-negative bacteria than administering the antibiotic alone.

31. A method for preventing or treating a bacterial infection caused by at least one species of Gram-negative bacteria, comprising co-administering to a subject diagnosed with, at risk for, or exhibiting symptoms of a bacterial infection, a combination of a first amount of a pharmaceutical composition according to claim 1 and a second amount of an antibiotic suitable for the treatment of Gram-negative bacterial infection, wherein the first and the second amounts together are effective for preventing or treating the bacterial infection.

32. The method of claim 31, wherein the antibiotic is selected from one or more of azithromycin, aztreonam, fosfomycin, ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

33. (canceled)

34. A method for augmenting the efficacy of an antibiotic suitable for the treatment of a bacterial infection caused by at least one species of Gram-negative bacteria, comprising co-administering the antibiotic in combination with a pharmaceutical composition according to claim 1,
wherein administration of the combination is more effective in inhibiting growth, reducing the population, or killing the Gram-negative bacteria than administration of either the antibiotic or the pharmaceutical composition individually.
